SEQUENCE LISTING (63 sequences)

SEQ ID NO: 1 - DNA, 369 bp, synthetic construct
  source 1..369: mol_type = other DNA
agcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgc

SEQ ID NO: 2 - protein, 123 aa, synthetic construct
SSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDC

SEQ ID NO: 3 - DNA, 339 bp, synthetic construct
  source 1..339: mol_type = other DNA
catctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 4 - protein, 113 aa, synthetic construct
HLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 5 - DNA, 543 bp, synthetic construct
  source 1..543: mol_type = other DNA
cagcccaagctgagaacaagattcaacctgctgggcagatacctgctgatcgcccaccccaagctgttcccccaagtgttcagcgccaagggcatcgtgctggccctggtgagcacccacagacaagtgaagagagccgcccaagccctggatcagtacaagctgagacaagcaagcatccacagcaaccacgtggctagaaccgacgaggccatgcccggcagatttcaagagctggacagcagactggcaagcggcggaccctccgagggtattagcagcgagcccgtcagtgccgccaccactcagacgctggagggcgacagcagcgaggtgggcgatcagatcaacagcaagcgggccaagcagatggccgaaaagtcaccccccgaagcgagcgctaaggctcagcccatcaagggcgcccgaagagcccccgacagcagcagcgagttcggcaaggtgacaagaatgaacagacagttcagacaagcctacggcagcatcctgaagagaggccctagaagcttccacagactgcac

SEQ ID NO: 6 - protein, 181 aa, synthetic construct
QPKLRTRFNLLGRYLLIAHPKLFPQVFSAKGIVLALVSTHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMNRQFRQAYGSILKRGPRSFHRLH

SEQ ID NO: 7 - DNA, 273 bp, synthetic construct
  source 1..273: mol_type = other DNA
cctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctg

SEQ ID NO: 8 - protein, 91 aa, synthetic construct
PSSKSDRYYKIVKPLWTSFIQSVSYSKTKKIFKSHLKSSRNSTSVKKKSSRNQPFREILCKKLHIPLKAQNDLDISRIKRGNTTLESTDTL

SEQ ID NO: 9 - DNA, 735 bp, synthetic construct
  source 1..735: mol_type = other DNA
atggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcc

SEQ ID NO: 10 - protein, 245 aa, synthetic construct
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 11 - DNA, 747 bp, synthetic construct
  source 1..747: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgcc

SEQ ID NO: 12 - protein, 249 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSA

SEQ ID NO: 13 - DNA, 729 bp, synthetic construct
  source 1..729: mol_type = other DNA
atgagccgacaggtgcaactgcaacagtcagggccagaactgaagaaaccgggcgaaaccgtcaaaataagttgtaaggcttccggctaccctttcaccaactatgggatgaactgggtaaagcaggcaccaggacaagggctgaaatggatggggtggatcaatacctccactggcgagtctacattcgcggacgatttcaaggggaggtttgatttctcattagagacttccgccaacacggcatacctacagatcaataatttgaagtccgaagacatggccacctacttttgcgctagatgggaggtgtatcacggctacgtgccctattggggtcaggggacaaccgtcaccgtgtctagcggtggaggcgggtccggcggaggcggaagcggtggcggcggatcagacattcagcttacccagagccacaaattcctgagcactagcgtcggcgacagggtgtcaattacttgcaaggcatctcaggatgtttataacgcggtggcctggtatcagcaaaaacctggtcagtcgccaaaactgctcatttactccgcttctagccgctacacaggggttcctagtcggttcacaggttccggaagtggacccgatttcacttttacaatctcgagtgtacaggccgaggacctcgccgtgtacttttgtcagcaacatttcagaaccccctttacgtttggatctggcacaaagctcgagatc

SEQ ID NO: 14 - protein, 243 aa, synthetic construct
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEI

SEQ ID NO: 15 - DNA, 1095 bp, synthetic construct
  source 1..1095: mol_type = other DNA
atggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatcccatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 16 - protein, 365 aa, synthetic construct
MDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLGTVSSRPGGGGSHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 17 - DNA, 1899 bp, synthetic construct
  source 1..1899: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatcccatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 18 - protein, 633 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 19 - DNA, 1929 bp, synthetic construct
  source 1..1929: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgc

SEQ ID NO: 20 - protein, 643 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDC

SEQ ID NO: 21 - DNA, 1833 bp, synthetic construct
  source 1..1833: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatcccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctg

SEQ ID NO: 22 - protein, 611 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSPSSKSDRYYKIVKPLWTSFIQSVSYSKTKKIFKSHLKSSRNSTSVKKKSSRNQPFREILCKKLHIPLKAQNDLDISRIKRGNTTLESTDTL

SEQ ID NO: 23 - DNA, 2085 bp, synthetic construct
  source 1..2085: mol_type = other DNA
atgagccgacaggtgcaactgcaacagtcagggccagaactgaagaaaccgggcgaaaccgtcaaaataagttgtaaggcttccggctaccctttcaccaactatgggatgaactgggtaaagcaggcaccaggacaagggctgaaatggatggggtggatcaatacctccactggcgagtctacattcgcggacgatttcaaggggaggtttgatttctcattagagacttccgccaacacggcatacctacagatcaataatttgaagtccgaagacatggccacctacttttgcgctagatgggaggtgtatcacggctacgtgccctattggggtcaggggacaaccgtcaccgtgtctagcggtggaggcgggtccggcggaggcggaagcggtggcggcggatcagacattcagcttacccagagccacaaattcctgagcactagcgtcggcgacagggtgtcaattacttgcaaggcatctcaggatgtttataacgcggtggcctggtatcagcaaaaacctggtcagtcgccaaaactgctcatttactccgcttctagccgctacacaggggttcctagtcggttcacaggttccggaagtggacccgatttcacttttacaatctcgagtgtacaggccgaggacctcgccgtgtacttttgtcagcaacatttcagaaccccctttacgtttggatctggcacaaagctcgagatcgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatcccagcccaagctgagaacaagattcaacctgctgggcagatacctgctgatcgcccaccccaagctgttcccccaagtgttcagcgccaagggcatcgtgctggccctggtgagcacccacagacaagtgaagagagccgcccaagccctggatcagtacaagctgagacaagcaagcatccacagcaaccacgtggctagaaccgacgaggccatgcccggcagatttcaagagctggacagcagactggcaagcggcggaccctccgagggtattagcagcgagcccgtcagtgccgccaccactcagacgctggagggcgacagcagcgaggtgggcgatcagatcaacagcaagcgggccaagcagatggccgaaaagtcaccccccgaagcgagcgctaaggctcagcccatcaagggcgcccgaagagcccccgacagcagcagcgagttcggcaaggtgacaagaatgaacagacagttcagacaagcctacggcagcatcctgaagagaggccctagaagcttccacagactgcac

SEQ ID NO: 24 - protein, 695 aa, synthetic construct
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSQPKLRTRFNLLGRYLLIAHPKLFPQVFSAKGIVLALVSTHRQVKRAAQALDQYKLRQASIHSNHVARTDEAMPGRFQELDSRLASGGPSEGISSEPVSAATTQTLEGDSSEVGDQINSKRAKQMAEKSPPEASAKAQPIKGARRAPDSSSEFGKVTRMNRQFRQAYGSILKRGPRSFHRLH

SEQ ID NO: 25 - DNA, 714 bp, synthetic construct
  source 1..714: mol_type = other DNA
gacatccagatgatccagagcccggctagcctttctgtttctgtgggcgagactgtcaccatcacctgccgcgcttccgagaacatctatagcaacctggcttggttccagcagaagcaaggcaaaagccctcagctactggtgtacgcggccaccaacctggcggacggcgtgccctctaggttttcaggctcggggtccgggacccagtactccctgaagattaactccctccagtccgaagatttcggcatctactactgtcagcacttttggggcaccccccgcaccttcggaggtgggaccaagctggagatcaagggaggcggaggctccggtggtgggggtagtggtggcggtggctcccaggtgcagctgcagcagagcgccgtggagctggcccgccctggcgcctctgtaaagatgtcatgcaaggcttctggctacacgttcacctccttcactatgcattgggtcaagcagcgtcccgggggactcgagtggatcggctacattaatccatcaagcggctacaccgagtacaaccagaaatttaaggacaagaccacattgactgcggacaaatcctcgtcgacggcctacatgcagcttgattctctgaccagcgatgacagtgccgtctattactgcgtgcgcggatcgtcccgcggcttcgactattggggccagggcacattggtgaccgtgtccgca

SEQ ID NO: 26 - protein, 238 aa, synthetic construct
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIKGGGGSGGGGSGGGGSQVQLQQSAVELARPGASVKMSCKASGYTFTSFTMHWVKQRPGGLEWIGYINPSSGYTEYNQKFKDKTTLTADKSSSTAYMQLDSLTSDDSAVYYCVRGSSRGFDYWGQGTLVTVSA

SEQ ID NO: 27 - DNA, 981 bp, synthetic construct
  source 1..981: mol_type = other DNA
agcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctgcatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 28 - protein, 327 aa, synthetic construct
SSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTLHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 29 - DNA, 642 bp, synthetic construct
  source 1..642: mol_type = other DNA
agcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctg

SEQ ID NO: 30 - protein, 214 aa, synthetic construct
SSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTL

SEQ ID NO: 31 - DNA, 708 bp, synthetic construct
  source 1..708: mol_type = other DNA
agcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 32 - protein, 236 aa, synthetic construct
SSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 33 - DNA, 1437 bp, synthetic construct
  source 1..1437: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgcggccgcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 479 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAAAADYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 2082 bp, synthetic construct
  source 1..2082: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgcggccgcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctgcatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 36 - protein, 694 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAAAADYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTLHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 37 - DNA, 1749 bp, synthetic construct
  source 1..1749: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctgcatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 38 - protein, 583 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSARPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTLHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 39 - DNA, 1800 bp, synthetic construct
  source 1..1800: mol_type = other DNA
atgagcagacaggtgcaactgcaacagtcagggccagaactgaagaaaccgggagaaaccgtcaaaataagttgtaaggcttccggctaccctttcaccaactatgggatgaactgggtaaagcaggcaccaggacaagggctgaaatggatggggtggatcaatacctccactggcgagtctacattcgcggacgatttcaaggggaggtttgatttctcattagagacttccgccaacacggcatacctacagatcaataatttgaagtccgaagacatggccacctacttttgcgctagatgggaggtgtatcacggctacgtgccctattggggtcaggggacaaccgtcaccgtgtctagcggtggaggcgggtccggcggaggcggaagcggtggcggcggatcagacattcagcttacccagagccacaaattcctgagcactagcgtcggcgacagggtgtcaattacttgcaaggcatctcaggatgtttataacgcggtggcctggtatcagcaaaaacctggtcagtcgccaaaactgctcatttactccgcttctagccgctacacaggggttcctagtcggttcacaggttccggaagtggacccgatttcacttttacaatctcgagtgtacaggccgaggacctcgccgtgtacttttgtcagcaacatttcagaaccccctttacgtttggatctggcacaaagctcgagatcgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatccacttcagggggcgggggatccctcgactccgggcaggtattactagagagcaacatcaaggtgctgcccacttggtcaaccccggtgcaacccatggcgctcattgttctggggggagtggccggactgctgctttttattggcctgggcatcttcttttgcgtccgatgtagacacagaaggaggcaggcagaacgcatgagtcagataaaacggctcttgtctgagaagaagacatgccagtgcccacatcggttccaaaaaacctgtagccctatc

SEQ ID NO: 40 - protein, 600 aa, synthetic construct
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 41 - DNA, 2523 bp, synthetic construct
  source 1..2523: mol_type = other DNA
atgagcagacaggtgcaactgcaacagtcagggccagaactgaagaaaccgggcgaaaccgtcaaaataagttgtaaggcttccggctaccctttcaccaactatgggatgaactgggtaaagcaggcaccaggacaagggctgaaatggatggggtggatcaatacctccactggcgagtctacattcgcggacgatttcaaggggaggtttgatttctcattagagacttccgccaacacggcatacctacagatcaataatttgaagtccgaagacatggccacctacttttgcgctagatgggaggtgtatcacggctacgtgccctattggggtcaggggacaaccgtcaccgtgtctagcggtggaggcgggtccggcggaggcggaagcggtggcggcggatcagacattcagcttacccagagccacaaattcctgagcactagcgtcggcgacagggtgtcaattacttgcaaggcatctcaggatgtttataacgcggtggcctggtatcagcaaaaacctggtcagtcgccaaaactgctcatttactccgcttctagccgctacacaggggttcctagtcggttcacaggttccggaagtggacccgatttcacttttacaatctcgagtgtacaggccgaggacctcgccgtgtacttttgtcagcaacatttcagaaccccctttacgtttggatctggcacaaagctggagatcgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctgcatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 42 - protein, 841 aa, synthetic construct
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTLHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 43 - DNA, 2541 bp, synthetic construct
  source 1..2541: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctgcatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 44 - protein, 847 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTLHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 45 - DNA, 1818 bp, synthetic construct
  source 1..1818: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatccacttcagggggcgggggatccctcgactccgggcaggtattactagagagcaacatcaaggtgctgcccacttggtcaaccccggtgcaacccatggcgctcattgttctggggggagtggccggactgctgctttttattggcctgggcatcttcttttgcgtccgatgtagacacagaaggaggcaggcagaacgcatgagtcagataaaacggctcttgtctgagaagaagacatgccagtgcccacatcggttccaaaaaacctgtagccctatc

SEQ ID NO: 46 - protein, 606 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 47 - DNA, 2202 bp, synthetic construct
  source 1..2202: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccctagcagcaagagcgacagatactacaagatcgtgaagcccctgtggacaagcttcattcagagcgtgagctacagcaagaccaagaagatcttcaagagccacctgaagagcagtaggaacagcacaagcgtcaaaaagaagagcagtcgcaatcagccgttcagagagatcctgtgcaagaagctgcacatccccctgaaggctcagaacgacctggacatcagcagaatcaagagaggcaacaccaccctggagagcaccgacaccctg

SEQ ID NO: 48 - protein, 734 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCPSSKSDRYYKIVKPLWTSFIQSVSYSKTKKIFKSHLKSSRNSTSVKKKSSRNQPFREILCKKLHIPLKAQNDLDISRIKRGNTTLESTDTL

SEQ ID NO: 49 - DNA, 2268 bp, synthetic construct
  source 1..2268: mol_type = other DNA
gacattcagctgacacagagccctagcagcctgagcgcaagcgtgggcgacagagtgaccatcacctgcagcgtgagcagcagcatcagcagcaacaacctgcactggtatcagcagaagcccggcaaggcccccaagccctggatctacggcacaagcaacctggcaagcggcgtgcctagcagattcagcggcagcggcagcggcaccgactacaccttcaccatcagcagcctgcagcccgaggacatcgccacctactactgtcagcagtggagcagctacccctacatgtacaccttcggccaaggaaccaaggtagagatcaagaggaccgtggccgggggcggtggttccggtggtggcggatctggaggcggcggatctgaagtgcaactggttgagtctggcgggggcgtcgtgcagcccggcagaagcctgagactgagctgcagcgcaagcggcttcactttctctggctacggtctgagctgggtgagacaagcccccggcaagggcctggagtgggtggccatgatcagcagcggcggcagctacacctactacgccgacagcgtgaagggcagattcgccatcagcagagacaacgccaagaacaccctgttcctgcagatggacagcctgagacccgaggacaccggcgtgtacttttgcgctagacatggcgacgatcccgcctggttcgcctactgggggcaaggcacccccgtgaccgtgagcagcgccgctagcgccgaagcagctgctaaagaggctgctgcaaaggaagctgctgccaaggcaatggatatccagatgacccaaagccctagcagcctgagcgccagcgtgggagatcgcgtaactatcacatgccgtgcttcccaggatatacgaaattaccttaattggtaccagcagaagcccggcaaggccccgaaactgctgatctactatacctcacgcctcgagtccggcgtaccctctaggttctccggaagtggttccggtaccgactatacactcactatctcttctctgcaaccagaagatttcgcgacgtattattgccagcaaggcaataccttaccctggaccttcgggcagggcacaaaggttgagattaaaggaggtggaggcagtgggggggggggctccggtgggggaggaagtgaagtgcagttggtggaaagtgggggcgggctggttcagccaggcgggtctctcaggctttcatgtgctgcatcggggtactcttttaccggctacacaatgaactgggtcagacaggccccaggaaaaggtctagagtgggtcgcactcattaacccttacaaaggagtcagtacttataatcagaagtttaaagacagatttacaatttccgtggacaagagcaagaacactgcatatctgcaaatgaactctttgcgggccgaggatacagccgtgtactattgtgctcggtcaggctactacggagacagcgattggtacttcgacgtctggggccagggcactctggtgaccgtgtcatcccggccggggggcgggggatccagcagccatcagctgagaagaaagcctagctacctggacagatacctgtgcctgagatacccccctagctacaaggccctgctgacaagaggcagaaaggcccatcagcacgtggcaagcctgagcggccaccaagacagacaagtgcccggcatggctagaatgagactggacgtgagactggccaagacaagaagcggcgagatcagaagcagcgcccaccactgcctggcccactggaagaagtgcgtgagaggcctgggcagcgaggccaaggaggaggcccctagaagcagcgtgaccgagaccgaggccgacggcaagatcaccccctggcccgacagcagagacctggacctgagcgactgccatctgaagtcttggaagagtagccgtgatagatacttcagagtggtgcacccccaccacgccctgaacaagatcagcaacagaagcctgagacagagacagatggacagacacgccaagatcaagagagcctacttcagcagccctagcttccccaacttcttcagcaccctgatcaacagatgcctgcagagaaagatgaccggcgagcccgacaacaacagatccactagcgtggagctaaccggcgaccccaacaagacaagaggcgcccccgaggccctgatggccaacagcggcgagccctggagcccgagctacctggggcccacaagcccc

SEQ ID NO: 50 - protein, 756 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSAASAEAAAKEAAAKEAAAKAMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSRPGGGGSSSHQLRRKPSYLDRYLCLRYPPSYKALLTRGRKAHQHVASLSGHQDRQVPGMARMRLDVRLAKTRSGEIRSSAHHCLAHWKKCVRGLGSEAKEEAPRSSVTETEADGKITPWPDSRDLDLSDCHLKSWKSSRDRYFRVVHPHHALNKISNRSLRQRQMDRHAKIKRAYFSSPSFPNFFSTLINRCLQRKMTGEPDNNRSTSVELTGDPNKTRGAPEALMANSGEPWSPSYLGPTSP

SEQ ID NO: 51 - protein, 5 aa, synthetic construct
GGGGS

SEQ ID NO: 52 - DNA, 732 bp, synthetic construct
  source 1..732: mol_type = other DNA
aacatcatgatgacgcagagtccatccagcctagccgtcagtgcaggcgagaaagtcacaatgacgtgtaagagctcacaatccgtgttgtattcaagcaatcaaaagaactatttggcatggtatcagcagaaacctgggcaatcacccaaacttctgatctactgggcatctacacgggaaagcggggtgcccgacaggttcaccggctccgggtctggtacagattttacactgacaatcagttctgtgcagcctgaggatctggctgtgtactactgtcaccagtacttatcgtcccacaccttcggaggaggaactaagctcgaaattaagcgtggcgggggagggagtggaggcggcggctccggcggcggcggaagccaactgcagcagccaggggccgagctcgttagaccgggaagcagcgtgaaactctcttgcaaggcctctggatacaccttcacccgctactggattcattgggtgaagcagcgaccaatccagggtctggagtggattggtaacatagacccctccgattccgaaactcactataatcagaagtttaaggacaaagctactctgactgtagataaaagcagtgggaccgcttatatgcagcttagctcactcacctcggaagactccgcggtttactattgcgccactgaggacctgtattacgccatggagtactggggccagggtacatcagtaaccgtctcttct

SEQ ID NO: 53 - protein, 244 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSS

SEQ ID NO: 54 - DNA, 744 bp, synthetic construct
  source 1..744: mol_type = other DNA
gacatcgtgatgacccaggctgcaccgtccgtgcccgtcactccgggcgagtccgtctcgatctcatgccgaagctccaaaaccctgctccattctaacggcaacacctacctgtactggttcttgcagagaccaggacagagccctcaggtcttaatctacaggatgtccaacctggcttccggcgtgcccaaccgcttctccggctccggctctgagactacgtttaccctccgtatctcgcgcgtagaggccgaggacgtggggatttattactgtatgcaacacctggagtacccctatacattcgggggaggtaccaagctggagatcaagggcggtggtggctcggggggcggcggttctggcggtggcggctcccaggtgcagctacagcagcctggtgctgagctggtgcgccccggcacctctgtgaagttgtcatgcaaggccagtggctacaccttcacatcctattggatgcactggattaagcagcgccctgaacagggccttgagtggatcggacgtatagacccgtacgattccgaaacccactacaacgagaaatttaaggacaaagcgatcctgtctgttgacaagagtagctcgactgcctacatccagctgagctctcttaccagcgatgactccgccgtgtactattgtagccgccgggacgccaagtacgatggctacgcgctggactactggggccagggcaccaccctgaccgtcagctcc

SEQ ID NO: 55 - protein, 248 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIKGGGGSGGGGSGGGGSQVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTTLTVSS

SEQ ID NO: 56 - DNA, 723 bp, synthetic construct
  source 1..723: mol_type = other DNA
gacattcagatgacacagagccctagcagcctgagcgcaagcgtgggcgacagagtgaccatcacctgccaagcaagccaagacatcagcaactacctgagctggtatcagcagaagcccggcaaggcccccaagctgctgatctactacaccaacctgctggccgagggcgtgcctagcagattcagcggcagcggcagcggcaccgacttcacctttaccattagcagtttgcagcccgaggacatcgccacctactattgccaacagtactacaactacagaacattcggacccggtactaaggtagacatcaaggggggcggcggcagcggcggcggtgggagcggcgggggaggttcagaagtgcagctggtggagagcggcgggggccttgtgaagcccggcggaagcctgagactgagctgcgccgcaagcggcttcaccttcagcgactacttcatgcactgggtgagacaagcccccggcaagggcctggagtgggtgggcgtgatcgacaccaagagcttcaactacgcaacctactacagcgacctggtgaagggcagattcaccatcagccgcgatgacagcaagaacaccctgtacctgcagatgaacagcctgaagaccgaggacaccgccgtgtactactgcaccgccaccatcgccgtgccctactacttcgactactggggccaaggcaccctggtgaccgtgagc

SEQ ID NO: 57 - protein, 241 aa, synthetic construct
DIQMTQSPSSLSASVGDRVTITCQASQDISNYLSWYQQKPGKAPKLLIYYTNLLAEGVPSRFSGSGSGTDFTFTISSLQPEDIATYYCQQYYNYRTFGPGTKVDIKGGGGSGGGGSGGGGSEVQLVESGGGLVKPGGSLRLSCAASGFTFSDYFMHWVRQAPGKGLEWVGVIDTKSFNYATYYSDLVKGRFTISRDDSKNTLYLQMNSLKTEDTAVYYCTATIAVPYYFDYWGQGTLVTVS

SEQ ID NO: 58 - DNA, 594 bp, synthetic construct
  source 1..594: mol_type = other DNA
gacacccaagagaaagaggtgagagccatggtgggcagcgacgtggagctgagctgcgcctgccccgagggcagcagattcgacctgaacgacgtgtacgtgtactggcagacaagcgagagcaagaccgtggtgacctaccacatccctcagaacagcagcctggagaacgtggacagcagatacagaaacagagccctgatgagccccgccggcatgctgagaggcgacttcagcctgagactgttcaacgtgaccccccaagacgagcagaagttccactgcctggtgctgtctcagagcctgggtttccaagaggtgctgtcagtggagcccgtggtgagcgcgccccacagccctagccaagacgagctgaccttcacctgcacaagcatcaacggctaccctagacccaacgtgtactggatcaacaagaccgacaacagcctgctggaccaagccctgcagaacgacaccgtgttcctgaacatgagaggcctgtacgacgtggtgagcgtgctgagaatcgctagaacccctagcgtgaacatcggctgctgcatcgagaacgtgctgctgcagcagaacctgacc

SEQ ID NO: 59 - protein, 198 aa, synthetic construct
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEPVVSAPHSPSQDELTFTCTSINGYPRPNVYWINKTDNSLLDQALQNDTVFLNMRGLYDVVSVLRIARTPSVNIGCCIENVLLQQNLT

SEQ ID NO: 60 - DNA, 300 bp, synthetic construct
  source 1..300: mol_type = other DNA
cagacaagcgtgagccctagcaaggtgatcctgcctagaggcggcagcgtgctggtgacctgcagcacaagctgcgatcagcctcagctgctgggcatcgagacccccctgagaaagaaggagctgctgctgcccggcaacaacagaaaggtgtacgagctgagcaacgtgcaagaggactctcagcccctgtgctggagcaactgccccgacgggcagagcgtggccaagaccttcctgaccgtgtactggacccccgagagagtggagctggcccccctgcctagctggcagcccgtg

SEQ ID NO: 61 - protein, 100 aa, synthetic construct
QTSVSPSKVILPRGGSVLVTCSTSCDQPQLLGIETPLRKKELLLPGNNRKVYELSNVQEDSQPLCWSNCPDGQSVAKTFLTVYWTPERVELAPLPSWQPV

SEQ ID NO: 62 - DNA, 396 bp, synthetic construct
  source 1..396: mol_type = other DNA
cccacaagcagcagcaccaagaagacacagctgcagctggagcacctgctgctggacctgcagatgatcctgaacggcatcaacaactacaagaaccccaagctgacaagaatgctgaccttcaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccaccttcatgtgcgagtacgccgacgagaccgccaccatcgtggagttcctgaacagatggatcaccttctctcagagcatcatcagcaccctgacc

SEQ ID NO: 63 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2025/0009881 A1
(43) Pub. Date: Jan. 9, 2025

(54) RECOMBINANT POLYPEPTIDES, RECOMBINANT NUCLEIC ACIDS ENCODING THE SAME, AND USES THEREOF IN TREATING CANCERS

(71) Applicant: RephImmune Biotechnology Inc., Taipei (TW)

(72) Inventor: Ming-Wei CHEN, Winchester, MA (US)

(21) Appl. No.: 18/751,398

(22) Filed: Jun. 24, 2024

Related U.S. Application Data
(60) Provisional application No. 63/524,892, filed on Jul. 4, 2023.

(51) Int. Cl. A61K 39/00; A61P 35/00; C07K 14/705; C07K 16/28; C07K 16/30

(52) U.S. Cl. CPC A61K 39/4633; A61K 39/4637; A61P 35/00; C07K 14/705; C07K 16/2803; C07K 16/2809; C07K 16/30; C07K 2317/622; C07K 2319/30

(57) ABSTRACT

Disclosed herein are recombinant polypeptides and recombinant nucleic acids encoding the same. According to some embodiments of the present disclosure, the recombinant polypeptide comprises a first bi-functional domain, and a first single-chain fragment variable (scFv) or a peptide linked to the N-terminus of the first bi-functional domain. Optionally, the recombinant polypeptide further comprises a second scFv linked to the N-terminus of the first scFv or peptide. Also disclosed herein are methods of treating cancers by using the immune cells expressing the recombinant polypeptides.

### CROSS-REFERENCE TO RELATED APPLICATIONS

This application relates to and claims the benefit of U.S. Provisional Application No. 63/524,892, filed Jul. 4, 2023; the content of the application is incorporated herein by reference in its entirety.

### SEQUENCE LISTING XML

The present application is being filed along with a Sequence Listing XML in electronic format. The Sequence Listing XML is provided as an XML file entitled P4339_SEQ_AF, created Jun. 6, 2024, which is 103 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to the treatment of cancers via novel cell therapy.

### 2. Description of Related Art

Cancer is a leading cause of death worldwide, accounting for nearly 10 million deaths in 2020, or nearly one in six deaths. One defining feature of cancer is the rapid growth of abnormal cells that develop uncontrollably and can invade or spread to any part of the body. The most common cancers include, breast, lung, colorectal, prostate, skin and gastric cancers. Signs and symptoms associated with cancer usually vary depending on what part of the body is affected. The general signs and symptoms of cancer include fatigue, lump, weight changes (e.g., unintended loss or gain), changes in bowel or bladder habits, difficulty swallowing, nausea, vomiting, fever, night sweats, bleeding, bruising, cough and/or trouble breathing. Symptoms of advanced cancer further include pain, weakened physical strength, and paralysis that affect the movement of cancer patients, resulting in depression, loss of independence and disruption of social relationships. In addition to the patients themselves, cancer also affects patients' family and friends. Many caregivers experienced a physical, mental, and emotional struggle. According to some reports, a cancer diagnosis even has a negative effect on the caregivers' health; for example, they may have higher rates of depression and weakened immune response, as well as cardiovascular diseases.

While different types of therapeutic approaches are currently available, including chemotherapy, surgery, radiation therapy, hormone therapy, targeted therapy, and most recently, immunotherapy and cell-based therapy, none of these treatments provides a satisfactory result. Cancer still poses significant health risks, and increases the personal, family, social and economic burden. In view of the foregoing, there is a continuing interest in developing a novel agent and method for treating cancer.

### SUMMARY

The following presents a simplified summary of the disclosure to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the present disclosure is directed to a recombinant polypeptide comprising a bi-functional domain, and a single-chain fragment variable (scFv) or peptide linked to the N-terminus of the bi-functional domain. According to the embodiments of the present disclosure, the bi-functional domain is capable of spanning cell membrane (i.e., serving as a transmembrane domain) and mediating signal transduction in cells (i.e., serving as an intracellular domain).

According to some embodiments of the present disclosure, the bi-functional domain comprises, from its N-terminus to C-terminus, in sequence, an intracellular loop 1 (ICL1), an ICL2, an ICL3 and a C-terminal region of a G protein-coupled receptor (GPCR), without comprising an extracellular domain and a transmembrane domain of the GPCR. According to some preferred embodiments, the bi-functional domain consists of the ICL1, ICL2, ICL3 and C-terminal region of the GPCR.

The GPCR is preferably a class A GPCR. According to some exemplary embodiments, the GPCR is cannabinoid receptor 2 (CNR2), hydroxycarboxylic acid receptor 2 (HCAR2), G-protein coupled receptor 84 (GPR84), or P2Y purinoceptor 14 (P2Y14).

According to one embodiment, the bi-functional domain comprises the ICL1, ICL2, ICL3 and C-terminal region of CNR2. In the embodiment, the CNR2-derived bi-functional domain comprises the amino acid sequence of SEQ ID NO: 2.

According to another embodiment, the bi-functional domain comprises the ICL1, ICL2, ICL3 and C-terminal region of HCAR2. In the embodiment, the HCAR2-derived bi-functional domain comprises the amino acid sequence of SEQ ID NO: 4.

According to another embodiment, the bi-functional domain comprises the ICL1, ICL2, ICL3 and C-terminal region of GPR84. In the embodiment, the GPR84-derived bi-functional domain comprises the amino acid sequence of SEQ ID NO: 6.

According to still another embodiment, the bi-functional domain comprises the ICL1, ICL2, ICL3 and C-terminal region of P2Y14. In the embodiment, the P2Y14-derived bi-functional domain comprises the amino acid sequence of SEQ ID NO: 8.

According to certain embodiments of the present disclosure, the scFv is specific to CD3 or NKp46.

According to some preferred embodiments, the recombinant polypeptide further comprises a scFv specific to a tumor-associated antigen (TAA).

According to alternative embodiments of the present disclosure, the recombinant polypeptide comprises two bi-functional domains (i.e., a first and a second bi-functional domains), in which the second bi-functional domain is disposed at and connected to the C-terminus of the first bi-functional domain. In the embodiments, the first bi-functional domain comprises, from its N-terminus to C-terminus, in sequence, an ICL1, an ICL2, an ICL3 and a C-terminal region of a first GPCR; and the second bi-functional domain comprises, from its N-terminus to C-terminus, in sequence, an ICL1, an ICL2, an ICL3 and a C-terminal region of a second GPCR. Neither the first bi-functional domain nor the second bi-functional domain comprises an extracellular domain and a transmembrane domain of the GPCRs (i.e., the first and second GPCRs). According to some preferred embodiments, the first bi-functional domain consists of the ICL1, ICL2, ICL3 and C-terminal region of the first GPCR, and the second bi-functional domain consists of the ICL1, ICL2, ICL3 and C-terminal region of the second GPCR. Preferably, the second GPCR is different from the first GPCR.

In the preferred embodiments, each of the first and second GPCRs is a class A GPCR. According to some embodiments, the first and second GPCRs are independently selected from the group consisting of CNR2, HCAR2, GPR84 and P2Y14.

According to one embodiment, the first bi-functional domain comprises the ICL1, ICL2, ICL3 and C-terminal region of CNR2, and the second bi-functional domain comprises the ICL1, ICL2, ICL3 and C-terminal region of P2Y14. In the embodiment, the CNR2-derived bi-functional domain comprises the amino acid sequence of SEQ ID NO: 2, and the P2Y14-derived bi-functional domain comprises the amino acid sequence of SEQ ID NO: 8.

According to another embodiment, the first bi-functional domain comprises the ICL1, ICL2, ICL3 and C-terminal region of CNR2, and the second bi-functional domain comprises the ICL1, ICL2, ICL3 and C-terminal region of HCAR2. In the embodiment, the CNR2-derived bi-functional domain comprises the amino acid sequence of SEQ ID NO: 2, and the HCAR2-derived bi-functional domain comprises the amino acid sequence of SEQ ID NO: 4.

According to alternative embodiments of the present disclosure, the recombinant polypeptide comprises three bi-functional domains (i.e., a first, a second and a third bi-functional domains), in which the second bi-functional domain is disposed at and connected to the C-terminus of the first bi-functional domain, and the third bi-functional domain is disposed at and connected to the C-terminus of the second bi-functional domain. In the embodiments, the first bi-functional domain comprises, from its N-terminus to C-terminus, in sequence, an ICL1, an ICL2, an ICL3 and a C-terminal region of a first GPCR; the second bi-functional domain comprises, from its N-terminus to C-terminus, in sequence, an ICL1, an ICL2, an ICL3 and a C-terminal region of a second GPCR; and the third bi-functional domain comprises, from its N-terminus to C-terminus, in sequence, an ICL1, an ICL2, an ICL3 and a C-terminal region of a third GPCR. None of the first to the third bi-functional domains comprises an extracellular domain and a transmembrane domain of the GPCRs (i.e., the first, second the third GPCRs). According to some preferred embodiments, the first bi-functional domain consists of the ICL1, ICL2, ICL3 and C-terminal region of the first GPCR, the second bi-functional domain consists of the ICL1, ICL2, ICL3 and C-terminal region of the second GPCR, and the third bi-functional domain consists of the ICL1, ICL2, ICL3 and C-terminal region of the third GPCR. Preferably, the first, second and third GPCRs are different from one another.

In the preferred embodiments, each of the first, second and third GPCRs is a class A GPCR. According to certain embodiments, the first, second and third GPCRs are independently selected from the group consisting of CNR2, HCAR2, GPR84 and P2Y14.

According to one embodiment, the first bi-functional domain comprises the ICL1, ICL2, ICL3 and C-terminal region of CNR2, the second bi-functional domain comprises the ICL1, ICL2, ICL3 and C-terminal region of P2Y14, and the third bi-functional domain comprises the ICL1, ICL2, ICL3 and C-terminal region of HCAR2. In this embodiment, the CNR2-derived bi-functional domain comprises the amino acid sequence of SEQ ID NO: 2, the P2Y14-derived bi-functional domain comprises the amino acid sequence of SEQ ID NO: 8, and the HCAR2-derived bi-functional domain comprises the amino acid sequence of SEQ ID NO: 4.

Also disclosed herein are a recombinant nucleic acid encoding the recombinant polypeptide of the present disclosure, and an immune cell expressing the recombinant polypeptide.

According to certain embodiments of the present disclosure, the recombinant nucleic acid comprises a promoter, and a first and a second coding sequences operably linked to the promoter, wherein the first coding sequence encodes the scFv or peptide, and the second coding sequence is disposed downstream to the first coding sequence and encodes one or more bi-functional domains.

In some embodiments, the second coding sequence encodes one bi-functional domain, for example, the CNR2-, HCAR2-, GPR84-, or P2Y14-derived bi-functional domain. According to one exemplary embodiment, the second coding sequence encoding the CNR2-derived bi-functional domain (SEQ ID NO: 2) comprises the nucleotide sequence of SEQ ID NO: 1. According to one exemplary embodiment, the second coding sequence encoding the HCAR2-derived bi-functional domain (SEQ ID NO: 4) comprises the nucleotide sequence of SEQ ID NO: 3. According to another exemplary embodiment, the second coding sequence encoding the GPR84-derived bi-functional domain (SEQ ID NO: 6) comprises the nucleotide sequence of SEQ ID NO: 5. According to another exemplary embodiment, the second coding sequence encoding the P2Y14-derived bi-functional domain (SEQ ID NO: 8) comprises the nucleotide sequence of SEQ ID NO: 7.

In some embodiments, the second coding sequence encodes two bi-functional domain (i.e., a first and a second bi-functional domains), in which the second bi-functional domain is disposed at and connected to the C-terminus of the first bi-functional domain. According to one exemplary embodiment, the second coding sequence encoding a CNR2-derived bi-functional domain (SEQ ID NO: 2) and a P2Y14-derived bi-functional domain (SEQ ID NO: 8) comprises the nucleotide sequence of SEQ ID NO: 29. According to another exemplary embodiment, the second coding sequence encoding a CNR2-derived bi-functional domain (SEQ ID NO: 2) and a HCAR2-derived bi-functional domain (SEQ ID NO: 4) comprises the nucleotide sequence of SEQ ID NO: 31.

In certain embodiments, the second coding sequence encodes three bi-functional domains (i.e., a first, a second and a third bi-functional domains), in which the second bi-functional domain is disposed at and connected to the C-terminus of the first bi-functional domain, and the third bi-functional domain is disposed at and connected to the C-terminus of the second bi-functional domain. According to one exemplary embodiment, the second coding sequence encoding a CNR2-derived bi-functional domain (SEQ ID NO: 2), a P2Y14-derived bi-functional domain (SEQ ID NO: 8) and a HCAR2-derived bi-functional domain (SEQ ID NO: 4) comprises the nucleotide sequence of SEQ ID NO: 27.

Depending on desired purpose, the immune cell expressing the recombinant polypeptide may be a T cell, a natural killer (NK) cell, a B cell, a basophil, an eosinophil, a dendritic cell (DC) or a neutrophil. According to one exemplary embodiment, the immune cell is a T cell. According to another exemplary embodiment, the immune cell is a NK cell.

Another aspect of the present disclosure pertains to a method of treating cancer in a subject. The method comprises administering to the subject an effective amount of the immune cell of the present disclosure.

Examples of cancer treatable with the present immune cell and/or method include, but are not limited to, breast cancer, gastric cancer, colorectal cancer, gallbladder cancer, prostate cancer, cervical cancer, ovarian carcinoma, chronic or acute lymphocytic leukemia, bladder cancer, renal cancer, hepatocellular carcinoma, head and neck squamous cell carcinoma, glioblastoma, esophageal cancer, pancreatic cancer, oral cancer, lung cancer, melanoma, and lymphoma.

Among all embodiments of the present disclosure, the subject is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings.

FIG. 1 is a schematic diagram of BE-TM/IC polypeptide according to one embodiment of the present disclosure.

FIG. 2 is a schematic diagram of 2xBE-TM/IC polypeptide according to another embodiment of the present disclosure.

FIGS. 3A and 3B are schematic diagrams of 2xBE-multiTM/IC polypeptides according to another embodiment of the present disclosure.

FIG. 4 is the result of tumor killing assay depicting the cytotoxic effect of engineered T cells expressing FRa_CAR or CAR-delITAM-CPH according to Example 1.2 of the present disclosure.

FIG. 5 is the result of tumor killing assay depicting the cytotoxic effect of engineered T cells expressing FRa_CAR or FRa-CPHTM/IC according to Example 1.2 of the present disclosure.

FIG. 6 is the result of tumor killing assay that depicts the cytotoxic effect of engineered T cells expressing HER2_TAC (Her2_TAC-T), HER2-CD3-syn GPR84 (Her2_syn GPR84), or HER2-CD3-syn CPH (Her2_syn CPH) according to Example 1.3 of the present disclosure.

FIGS. 7A and 7B are analytic results respectively depicting the cytotoxic effect of specified engineered T cells on cancer cells according to Example 1.3 of the present disclosure. FRa_syn CPH: Engineered T cells expressing FRa_CD3_syn CPH. FRa_TAC-T: Engineered T cells expressing FRa_TAC. FRa_CAR-T: Engineered T cells expressing FRa_CAR.

FIGS. 8A to 8C are histograms respectively depicting the expression levels of interleukin-2 (IL-2; FIG. 8A), interferon-gamma (IFN-γ; FIG. 8B), and tumor necrosis factor-alpha (TNF-α; FIG. 8C) in the supernatant of cultured cells according to Example 1.3 of the present disclosure, in which cancer cells were co-cultured with specified engineered T cells for 48 hours followed by the analysis of cytokine assays. FRa_syn CPH: Engineered T cells expressing FRa_CD3_syn CPH. FRa_TAC-T: Engineered T cells expressing FRa_TAC. FRa_CAR: Engineered T cells expressing FRa_CAR. **p<0.01; ***p<0.001; n.s. (not significant): p≥0.05.

FIG. 9 is a histogram depicting the cytotoxic effect of specified engineered T cells according to Example 1.3 of the present disclosure. FRa_CPH: Engineered T cells expressing FRa-CD3-syn CPH. FRa_CH: Engineered T cells expressing FRa-CD3-syn CH. FRa_CP: Engineered T cells expressing FRa-CD3-syn CP. FRa_C: Engineered T cells expressing FRa_CD3_syn CNR2. FRa_H: Engineered T cells expressing FRa-CD3-syn HCAR2. FRa_P: Engineered T cells expressing FRa-CD3-syn P2Y14.

FIG. 10 is a line chart depicting the tumor volumes of mice administered with untransduced T cells, engineered T cells expressing FRa_TAC (FRa_TAC-T), or engineered T cells expressing FRa_CD3_syn CPH (FRa_syn CPH) according to Example 2 of the present disclosure. *p<0.05; **p<0.01.

FIG. 11 is a line chart depicting the probability of survival of mice administered with untransduced T cells, engineered T cells expressing FRa_TAC (FRa_TAC-T), or engineered T cells expressing FRa_CD3_syn CPH (FRa_syn CPH) according to Example 2 of the present disclosure.

In accordance with common practice, the various described features/elements are not drawn to scale but instead are drawn to best illustrate specific features/elements relevant to the present invention. Also, reference numerals and designations in the various drawings are used to indicate elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “recombinant nucleic acid” refers to a nucleic acid produced by recombinant DNA techniques, wherein the nucleotide sequence of the nucleic acid is not identical to that of any naturally occurring sequence. The term “recombinant polypeptide” refers to a polypeptide that is expressed and isolated from a cell or cell line transfected with an expression vector comprising the coding sequence of the polypeptide (e.g., the recombinant nucleic acid of the present disclosure), where said coding sequence is not naturally associated with the cell.

As used herein, the term “operably linked” refers to both expression control sequences that are contiguous with the gene of interest (e.g., the first and second coding sequences of the present disclosure) and expression control sequences (e.g., the promoter of the present disclosure) that act in trans or at a distance to control the gene of interest.

As discussed herein, minor variations in the amino acid sequences of polypeptides or in the nucleotide sequence of nucleic acids are contemplated as being encompassed by the presently disclosed and claimed inventive concept(s), providing that the variations in the amino acid sequence/nucleotide sequence maintain at least 85% sequence identity, such as at least 85%, 86%, 87%, 88%, 89%, 90%, 91%, 92%, 93%, 94%, 95%, 96%, 97%, 98% and 99% sequence identity. Polypeptides or nucleic acids of the present disclosure may be modified specifically to alter a feature of the polypeptide or nucleic acid unrelated to its physiological activity. For example, certain nucleotides can be changed without affect the physiological activity of the encoded polypeptide due to codon degeneracy (i.e., multiple codons can code for the same amino acid residue during protein synthesis; for example, the leucine residue can be encoded by the codon of “UUA”, “UUG”, “CUA”, “CUG”, “CUU” or “CUC”, and the serine residue can be encoded by the codon of “UCA”, “UCG”, “UCC”, “UCU”, “AGU” or “AGC”). Additionally, certain amino acids can be changed and/or deleted without affecting the physiological activity of the polypeptide in this study. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site.

“Percentage (%) sequence identity” is defined as the percentage of amino acid residues/nucleotides in a candidate sequence that are identical with the amino acid residues/nucleotides in the specific polypeptide/nucleic acid, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences/nucleic acids was carried out by computer program Blastp (protein-protein BLAST)/Blastn (nucleotide-nucleotide BLAST) provided online by Nation Center for Biotechnology Information (NCBI). The percentage amino acid sequence/nucleic acid identity of a given amino acid sequence/nucleic acid A to a given amino acid sequence/nucleic acid B (which can alternatively be phrased as a given amino acid sequence/nucleic acid A that has a certain % amino acid sequence/nucleic acid identity to a given amino acid sequence/nucleic acid B) is calculated by the formula as follows:

$$\frac{X}{Y} \times 100 \%$$

where X is the number of amino acid residues/nucleic acids scored as identical matches by the sequence alignment program BLAST in that program's alignment of A and B, and where Y is the total number of amino acid residues/nucleic acids in A or B, whichever is shorter.

As used herein, the terms “link” and “connect” are used interchangeably to refer to any means of connecting two components either via direct linkage or via indirect linkage between two components.

The term “administered”, “administering” or “administration” are used interchangeably herein to refer a mode of delivery, including, without limitation, intratumorally, intravenously, intraarterially, or intraperitoneally delivering an agent (e.g., the immune cell) of the present invention.

As used herein, the terms “treat,” “treating” and “treatment” are interchangeable, and encompass partially or completely preventing, ameliorating, mitigating and/or managing a symptom, a secondary disorder or a condition associated with cancer. The term “treating” as used herein refers to application or administration of the present immune cells to a subject, who has a symptom, a secondary disorder or a condition associated with cancer, with the purpose to alleviate, ameliorate, relieve, delay onset of, inhibit progression of, reduce severity of, and/or reduce incidence of one or more symptoms, secondary disorders or features associated with cancer partially or completely. Symptoms, secondary disorders, and/or conditions associated with cancer include, but are not limited to, fatigue, lump, weight changes (e.g., unintended loss or gain), changes in bowel or bladder habits, difficulty swallowing, nausea, vomiting, fever, night sweats, bleeding, bruising, cough, trouble breathing, pain, weakened physical strength, and paralysis. Treatment may be administered to a subject who exhibits only early signs of such symptoms, disorder, and/or condition for the purpose of decreasing the risk of developing the symptoms, secondary disorders, and/or conditions associated with cancer. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designates the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed. Effective amounts may be expressed, for example, in cell numbers of body weight (cells/Kg), cell numbers of body surface area (cells/m²), or cell numbers per subject (cells/subject). Persons having ordinary skills could calculate the human equivalent dose (HED) for the medicament (such as the present immune cell) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the immune cell and/or method of the present invention. The term “subject” is intended to refer to both the male and female gender unless one gender is specifically indicated.

### II. Description of the Invention

GPCRs, also known as “seven-transmembrane domain receptors”, are membrane-bound receptors coupled with heterotrimeric G proteins that regulate responses to sensory stimuli such as light, odors, hormones, cytokines, or neurotransmitters. In structure, GPCR comprises seven (H1-H7) transmembrane alpha-helices connected by three extracellular loops (ECL1, ECL2, ECL3) and three intracellular loops (ICL1, ICL2, ICL3) with an extracellular N-terminal region (also known as “N-terminal tail”) and an intracellular C-terminal region (also known as “C-terminal tail”). GPCRs are categorized into six classes based on sequence homology and functional similarity, including class A: rhodopsin-like receptors, class B: secretin family, class C: metabotropic glutamate receptors, class D: fungal mating pheromone receptors, class E: cAMP receptors, and class F: frizzled (FZD) and smoothened (SMO) receptors.

The present disclosure is based, at least in part, on the unexpected discovery that a reassembled peptide comprising the ICL1, ICL2, ICL3 and C-terminal region of class A GPCR may serve as a bi-functional domain (hereinafter as “TM/IC domain”) capable of traversing/spanning cell membrane (i.e., serving as a transmembrane domain) and mediating signal transduction in cells (i.e., serving as an intracellular domain). The novel TM/IC domain is useful in coupling with one or more extracellular proteins (e.g., one or more scFvs or peptides) and expressing the extracellular protein(s) on the surface of cells (e.g., immune cells). According to some exemplary embodiments of the present disclosure, the novel TM/IC domain is linked to two extracellular proteins (e.g., two scFvs, two peptides, or one scFv and one peptide), which serve as binding elements (BEs) and are respectively specific to TAA and immune cell-associated molecule (IAM; i.e., the molecule expressed on the surface of immune cell and preferably associated with the activation of the immune cell). The thus-formed BEs-TM/IC polypeptide is useful in redirecting immune cell specificity towards TAA-expressing tumor cells via the TAA-binding scFv/peptide, and then mediating immune cell activation and initiating the effector function of immune cell (e.g., proliferation, cytokine secretion and/or cytolysis) via the IAM-binding scFv/peptide and TM/IC domain. The novel TM/IC domain thus provides a novel strategy to construct different types of chimeric immune cell receptors (e.g., chimeric T cell receptor or chimeric NK cell receptor) for use in cell therapy.

Accordingly, the present disclosure provides several recombinant polypeptides, including BE-TM/IC polypeptide, 2xBE-TM/IC polypeptide, and 2xBE-multiTM/IC polypeptide, which respectively comprise one or two TM/IC domains serving as the bi-functional domain for anchoring the polypeptide to cell membrane, and one or more BEs serving as targeting domain for interacting with target molecule(s). Also disclosed herein are nucleic acids for expressing the recombinant polypeptides, immune cells having the recombinant polypeptide expressed thereon, and methods of treating diseases (e.g., cancers) by using the immune cells.

### (i) BE-TM/IC Polypeptides and Nucleic Acids Encoding the Same

The first aspect of the present disclosure is directed to a BE-TM/IC polypeptide, and a nucleic acid encoding the BE-TM/IC polypeptide.

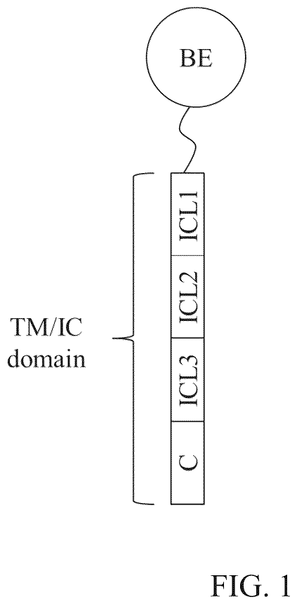

Reference is now made to FIG. 1, which depicts the schematic diagram of the BE-TM/IC polypeptide in accordance with one embodiment of the present disclosure. In structure, the BE-TM/IC polypeptide comprises one TM/IC domain and one binding element (BE) linked to the N-terminus of the TM/IC domain, in which the TM/IC domain comprises, from its N-terminus to C-terminus, in sequence, the ICL1, ICL2, ICL3 and C-terminal region (C) of a GPCR. Depending on desired purpose, the BE may be a scFv or a peptide, in which in the case when the BE is a scFv, it has a general structure known in the art, and comprises a heavy chain variable domain (VH) and a light chain variable domain (VL). After expressed in cells (e.g., immune cells), the BE (i.e., scFv or peptide) functions as an extracellular domain that recognizes and targets to a corresponding molecule, and the TM/IC domain functions as a bi-functional domain that anchors the BE to cell membrane and mediates the signal transduced therefrom.

According to some embodiments of the present disclosure, the BE is a scFv specific to a receptor, co-receptor, co-stimulatory molecule, or cell adhesion molecule of an immune cell (e.g., a T cell, NK cell, B cell, basophil, cosinophil, DC or neutrophil). In one embodiment, the BE is a scFv specific to CD3, a protein complex and T cell co-receptor involved in T cell activation. According to one example of the present disclosure, the anti-CD3 scFv comprises the amino acid sequence of SEQ ID NO: 10. In another embodiment, the BE is a scFv specific to NKp46, a major NK cell-activating receptor. According to one example of the present disclosure, the anti-NKp46 scFv comprises the amino acid sequence of SEQ ID NO: 26. In another embodiment, the BE is a scFv specific to CD2, a cell adhesion molecule regulating the activation of T cells and NK cells. According to one example of the present disclosure, the anti-CD2 scFv comprises the amino acid sequence of SEQ ID NO: 53 or 55. In still another embodiment, the BE is a scFv specific to inducible costimulator (ICOS), a co-stimulatory molecule associated with T cell activation. According to one example of the present disclosure, the anti-ICOS scFv comprises the amino acid sequence of SEQ ID NO: 57.

According to certain embodiments of the present disclosure, the BE is a peptide recognized by an immunomodulatory receptor or molecule. In one embodiment, the BE is an inducible costimulator-ligand (ICOS-L). According to one example of the present disclosure, the ICOS-L peptide comprises the amino acid sequence of SEQ ID NO: 59. In another embodiment, the BE is a cell adhesion molecule 1 (CAMD1). According to one example of the present disclosure, the CAMD1 peptide comprises the amino acid sequence of SEQ ID NO: 61. In another embodiment, the BE is an IL-2 binder. According to one example of the present disclosure, the IL-2 binder comprises the amino acid sequence of SEQ ID NO: 63. As could be appreciated, the BE may vary with desired purpose, for example, the type of immune cell intended to be activated. A skilled artisan may choose a suitable BE for use in the present invention in accordance with the intended purpose.

Regarding the TM/IC domain of the BE-TM/IC polypeptide, it is characterized by having the intracellular domain (including ICL1, ICL2, ICL3 and C-terminal region) of the GPCR, without including the extracellular domain and transmembrane domain of the GPCR.

Depending on desired purpose, the ICL1, ICL2, ICL3 and C-terminal region of the TM/IC domain may be linked with or without a linker sequence. For example, the present TM/IC domain may be in the form of “ICL1-linker-ICL2-linker-ICL3-linker-C-terminal region”, in which the ICL1, ICL2, ICL3 and C-terminal region are linked via a suitable linker sequence. Alternatively, the present TM/IC domain may be in the form of “ICL1-ICL2-ICL3-C-terminal region”, in which the ICL1, ICL2, ICL3 and C-terminal region are linked without a linker sequence. According to some preferred embodiment, the present TM/IC domain consists of the ICL1, ICL2, ICL3 and C-terminal region of the GPCR.

Preferably, the GPCR is a class A GPCR; for example, CNR2, HCAR2, GPR84, or P2Y14.

According to some embodiments, the TM/IC domain designated as “syn CNR2” comprises the ICL1 (a.a. 60-71), ICL2 (a.a. 130-149), ICL3 (a.a. 215-246) and C-terminal region (a.a 302-360) of CNR2. In one exemplary embodiment, the syn CNR2 is in the form the “ICL1-ICL2-ICL3-C-terminal region” and comprises the amino acid sequence of

(SEQ ID NO: 2)
“SSHQLRRKPSYLDRYLCLRYPPSYKALLTRGRKAHQHVASLSGHQD
RQVPGMARMRLDVRLAKTRSGEIRSSAHHCLAHWKKCVRGLGSEAKE
EAPRSSVTETEADGKITPWPDSRDLDLSDC”.

According to some embodiments, the TM/IC domain designated as “syn HCAR2” comprises the ICL1 (a.a. 55-63), ICL2 (a.a. 124-142), ICL3 (a.a. 214-229) and C-terminal region (a.a. 295-363) of HCAR2. In one exemplary embodiment, the syn HCAR2 is in the form the “ICL1-ICL2-ICL3-C-terminal region” and comprises the amino acid sequence of

(SEQ ID NO: 4)
“HLKSWKSSRDRYFRVVHPHHALNKISNRSLRQRQMDRHAKIKRAYF
SSPSFPNFFSTLINRCLQRKMTGEPDNNRSTSVELTGDPNKTRGAPE
ALMANSGEPWSPSYLGPTSP”.

According to certain embodiments, the TM/IC domain designated as “syn GPR84” comprises the ICL1 (a.a. 48-57), ICL2 (a.a. 116-144), ICL3 (a.a. 202-320) and C-terminal region (a.a. 374-396) of GPR84. In one exemplary embodiment, the syn GPR84 is in the form the “ICL1-ICL2-ICL3-C-terminal region” and comprises the amino acid sequence of

(SEQ ID NO: 6)
“QPKLRTRFNLLGRYLLIAHPKLFPQVFSAKGIVLALVSTHRQVKRA
AQALDQYKLRQASIHSNHVARTDEAMPGRFQELDSRLASGGPSEGIS
SEPVSAATTQTLEGDSSEVGDQINSKRAKQMAEKSPPEASAKAQPIK
GARRAPDSSSEFGKVTRMNRQFRQAYGSILKRGPRSFHRLH”.

According to certain embodiments, the TM/IC domain designated as “syn P2Y14” comprises the ICL1 (a.a. 51-55), ICL2 (a.a. 118-139), ICL3 (a.a. 210-234) and C-terminal region (a.a. 300-338) of P2Y14. In one exemplary embodiment, the syn P2Y14 is in the form the “ICL1-ICL2-ICL3-C-terminal region” and comprises the amino acid sequence of

(SEQ ID NO: 8)
″PSSKSDRYYKIVKPLWTSFIQSVSYSKTKKIFKSHLKSSRNSTSVK
KKSSRNQPFREILCKKLHIPLKAQNDLDISRIKRGNTTLESTDTL″.

According to some exemplary embodiments, for the purpose of activating T cells, the present BE-TM/IC polypeptide comprises an anti-CD3 scFv (e.g., the anti-CD3 scFv of SEQ ID NO: 10), and a TM/IC domain (for example, any of the syn CNR2, syn HCAR2, syn GPR84 and syn P2Y14). Depending on desired purpose, the anti-CD3 scFv may alternatively comprise a Y177T mutation; for example, the anti-CD3 scFv (huUCHT1 Y177T) described in U.S. Patent Application NO. US 2020/0392247. Alternatively, the present BE-TM/IC polypeptide may comprise an anti-NKp46 scFv (e.g., an anti-NKp46 scFv of SEQ ID NO: 26), and a TM/IC domain (for example, any of the syn CNR2, syn HCAR2, syn GPR84 and syn P2Y14) for the purpose of activating NK cells.

Preferably, the BE is linked to the TM/IC domain via a linker. According to some preferred embodiments, the linker comprises the amino acid residues independently selected from the group consisting of glycine (G) and serine(S) residues. In one exemplary embodiment, the linker comprises the amino acid sequence of “GGGGS” (SEQ ID NO: 51). The linker may be any linkers known to conjugate two peptides. A skilled artisan may select a suitable linker to link the present BE and TM/IC domain in accordance with intended purposes.

As an example, the amino acid sequence of a BE-TM/IC polypeptide referred to as “CD3-syn HCAR2” that comprises an anti-CD3 scFv (SEQ ID NO: 10) and the syn HCAR2 (SEQ ID NO: 4) is provided below, in which the anti-CD3 scFv is marked in italic, the syn HCAR2 is bold, and a linker sequence is disposed therebetween.

(Amino acid sequence of CD3-syn HCAR2)
SEQ ID NO: 16
MDIQMTQSPSSLSASVGDRVTITCRASQDIRNYLNWYQQKPGKAPKL
LIYYTSRLESGVPSRFSGSGSGTDYTLTISSLQPEDFATYYCQQGNT
LPWTFGQGTKVEIKGGGGSGGGGSGGGGSEVQLVESGGGLVQPGGSL
RLSCAASGYSFTGYTMNWVRQAPGKGLEWVALINPYKGVSTYNQKFK
DRFTISVDKSKNTAYLQMNSLRAEDTAVYYCARSGYYGDSDWYFDVW
GQGTLGTVSSRPGGGGSHLKSWKSSRDRYFRVVHPHHALNKISNRSL
RQRQMDRHAKIKRAYFSSPSFPNFFSTLINRCLQRKMTGEPDNNRST
SVELTGDPNKTRGAPEALMANSGEPWSPSYLGPTSP

Also disclosed herein are nucleic acids encoding the present BE-TM/IC polypeptides. The nucleic acid comprises a promoter, a BE-coding sequence (a first coding sequence) and a TM/IC-coding sequence (a second coding sequence), in which the BE-coding and TM/IC-coding sequences are operably linked to the promoter, and the TM/IC-coding sequence is disposed downstream to the BE-coding sequence.

The promoter may be an inducible promoter or a constitutive promoter. As known in the art, the term “inducible promoter” refers to a promoter whose performance is not conditioned to endogenous factors but to environmental conditions and external stimuli that can be artificially controlled. Examples of the inducible promoter suitable for use in driving the expression of the first and second coding sequences of the present nucleic acid include, but are not limited to, heat shock inducible promoter, metallothionein promoter, ecdysone-inducible promoter, FKBP dimerization inducible promoter, Gal4-estrogen receptor fusion protein regulated promoter, steroid inducible promoter, streptogramin responsive promoter, and tetracycline regulated promoter. Regarding the constitutive promoter, it is a promoter whose activity is maintained at a relatively constant level in all cells of an organism with little or no regard to cell environmental conditions (such as the concentration of a substrate). Non-limiting example of the constitutive promoter suitable for use in the present nucleic acid include cytomegalovirus (CMV) promoter, rous sarcoma virus (RSV) promoter, simian virus (SV40) promoter, mouse mammary tumor virus (MMTV) promoter, phosphoglycerate kinase (PGK) promoter, chicken beta-active promoter, elongation factor 1-alpha (EF1-α) promoter, human H1 promoter, and U6 promoter. According to one exemplary embodiment, the promoter is EF1-α promoter.

In some embodiments, the BE-coding sequence encodes an anti-CD3 scFv, for example, the anti-CD3 scFv of SEQ ID NO: 10, in which the anti-CD3 scFv-coding sequence comprises a nucleotide sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 9; preferably, at least 90% identical to SEQ ID NO: 9; more preferably, at least 95% identical to SEQ ID NO: 9. In one example of the present disclosure, the anti-CD3 scFv-coding sequence comprises the nucleotide sequence of SEQ ID NO: 9, i.e., comprising a nucleotide sequence 100% identical to SEQ ID NO: 9.

In some embodiments, the BE-coding sequence encodes an anti-NKp46 scFv, for example, the anti-NKp46 scFv of SEQ ID NO: 26, in which the anti-NKp46 scFv-coding sequence comprises a nucleotide sequence at least 85% identical to SEQ ID NO: 25; preferably, at least 90% identical to SEQ ID NO: 25; more preferably, at least 95% identical to SEQ ID NO: 25. In one example of the present disclosure, the anti-NKp46 scFv-coding sequence comprises the nucleotide sequence of SEQ ID NO: 25.

In certain embodiments, the BE-coding sequence encode an anti-CD2 scFv, for example, the anti-CD2 scFv of SEQ ID NO: 53 or 55, in which the anti-CD2 scFv-coding sequence comprises a nucleotide sequence at least 85% identical to SEQ ID NO: 52 or 54; preferably, at least 90% identical to SEQ ID NO: 52 or 54; more preferably, at least 95% identical to SEQ ID NO: 52 or 54. In one example of the present disclosure, the BE-coding sequence encoding the anti-CD2 scFv of SEQ ID NO: 53 comprises the nucleotide sequence of SEQ ID NO: 52. In another example of the present disclosure, the BE-coding sequence encoding the anti-CD2 scFv of SEQ ID NO: 55 comprises the nucleotide sequence of SEQ ID NO: 54.

In certain embodiments, the BE-coding sequence encodes an anti-ICOS scFv, for example, the anti-ICOS scFv of SEQ ID NO: 57, in which the anti-ICOS scFv-coding sequence comprises a nucleotide sequence at least 85% identical to SEQ ID NO: 56; preferably, at least 90% identical to SEQ ID NO: 56; more preferably, at least 95% identical to SEQ ID NO: 56. In one example of the present disclosure, the BE-coding sequence encoding the anti-ICOS scFv of SEQ ID NO: 57 comprises the nucleotide sequence of SEQ ID NO: 56.

In various embodiments, the BE-coding sequence encodes an ICOS-L peptide, for example, the ICOS-L peptide of SEQ ID NO: 59, in which the ICOS-L-coding sequence comprises a nucleotide sequence at least 85% identical to SEQ ID NO: 58; preferably, at least 90% identical to SEQ ID NO: 58; more preferably, at least 95% identical to SEQ ID NO: 58. In one example of the present disclosure, the BE-coding sequence encoding the ICOS-L of SEQ ID NO: 59 comprises the nucleotide sequence of SEQ ID NO: 58.

In some embodiments, the BE-coding sequence encodes a CAMD1 peptide, for example, the CAMD1 peptide of SEQ ID NO: 61, in which the CAMD1-coding sequence comprises a nucleotide sequence at least 85% identical to SEQ ID NO: 60; preferably, at least 90% identical to SEQ ID NO: 60; more preferably, at least 95% identical to SEQ ID NO: 60. In one example of the present disclosure, the BE-coding sequence encoding the CAMD1 peptide of SEQ ID NO: 61 comprises the nucleotide sequence of SEQ ID NO: 60.

In alternative embodiments, the BE-coding sequence encodes an IL-2 binder, for example, the IL-2 binder of SEQ ID NO: 63, in which the IL-2 binder-coding sequence comprises a nucleotide sequence at least 85% identical to SEQ ID NO: 62; preferably, at least 90% identical to SEQ ID NO: 62; more preferably, at least 95% identical to SEQ ID NO: 62. In one example of the present disclosure, the BE-coding sequence encoding the IL-2 binder of SEQ ID NO: 63 comprises the nucleotide sequence of SEQ ID NO: 62.

The TM/IC-coding sequence encodes the present TM/IC domain. According to certain embodiments, the TM/IC-coding sequence encodes the syn CNR2 (SEQ ID NO: 2), in which the syn CNR2-coding sequence comprises a nucleotide sequence at least 85% identical to SEQ ID NO: 1; preferably, at least 90% identical to SEQ ID NO: 1; more preferably, at least 95% identical to SEQ ID NO: 1. In one example of the present disclosure, the syn CNR2-coding sequence comprises the nucleotide sequence of SEQ ID NO: 1.

According to certain embodiments, the TM/IC-coding sequence encodes the syn HCAR2 (SEQ ID NO: 4), in which the syn HCAR2-coding sequence comprises a nucleotide sequence at least 85% identical to SEQ ID NO: 3; preferably, at least 90% identical to SEQ ID NO: 3; more preferably, at least 95% identical to SEQ ID NO: 3. In one example of the present disclosure, the syn HCAR2-coding sequence comprises the nucleotide sequence of SEQ ID NO: 3.

According to some embodiments, the TM/IC-coding sequence encodes the syn GPR84 (SEQ ID NO: 6), in which the syn GPR84-coding sequence comprises a nucleotide sequence at least 85% identical to SEQ ID NO: 5; preferably, at least 90% identical to SEQ ID NO: 5; more preferably, at least 95% identical to SEQ ID NO: 5. In one example of the present disclosure, the syn GPR84-coding sequence comprises the nucleotide sequence of SEQ ID NO: 5.

According to some embodiments, the TM/IC-coding sequence encodes the syn P2Y14 (SEQ ID NO: 8), in which the syn P2Y14-coding sequence comprises a nucleotide sequence at least 85% identical to SEQ ID NO: 7; preferably, at least 90% identical to SEQ ID NO: 7; more preferably, at least 95% identical to SEQ ID NO: 7. In one example of the present disclosure, the syn P2Y14-coding sequence comprises the nucleotide sequence of SEQ ID NO: 7.

As an example, the nucleotide sequence of the nucleic acid encoding the present CD3-syn HCAR2 (SEQ ID NO: 16) is provided below, in which the anti-CD3 scFv-coding sequence (SEQ ID NO: 9) is marked in italic, the syn HCAR2-coding sequence (SEQ ID NO: 3) is marked in bold, and a linker sequence is disposed therebetween.

(Nucleotide sequence of the nucleic acid encoding
CD3-syn HCAR2)
SEQ ID NO: 15
ATGGATATCCAGATGACCCAAAGCCCTAGCAGCCTGAGCGCCAGCGTGG
GAGATCGCGTAACTATCACATGCCGTGCTTCCCAGGATATACGAAATTA
CCTTAATTGGTACCAGCAGAAGCCCGGCAAGGCCCCGAAACTGCTGATC
TACTATACCTCACGCCTCGAGTCCGGCGTACCCTCTAGGTTCTCCGGAA
GTGGTTCCGGTACCGACTATACACTCACTATCTCTTCTCTGCAACCAGA
AGATTTCGCGACGTATTATTGCCAGCAAGGCAATACCTTACCCTGGACC
TTCGGGCAGGGCACAAAGGTTGAGATTAAAGGAGGTGGAGGCAGTGGGG
GGGGGGGGTCCGGTGGGGGAGGAAGTGAAGTGCAGTTGGTGGAAAGTGG
GGGCGGGCTGGTTCAGCCAGGCGGGTCTCTCAGGCTTTCATGTGCTGCA
TCGGGGTACTCTTTTACCGGCTACACAATGAACTGGGTCAGACAGGCCC
CAGGAAAAGGTCTAGAGTGGGTCGCACTCATTAACCCTTACAAAGGAGT
CAGTACTTATAATCAGAAGTTTAAAGACAGATTTACAATTTCCGTGGAC
AAGAGCAAGAACACTGCATATCTGCAAATGAACTCTTTGCGGGCCGAGG
ATACAGCCGTGTACTATTGTGCTCGGTCAGGCTACTACGGAGACAGCGA
TTGGTACTTCGACGTCTGGGGCCAGGGCACTCTGGTGACCGTGTCATCC
CGGCCGGGGGGCGGGGGATCCCATCTGAAGTCTTGGAAGAGTAGCCGTG
ATAGATACTTCAGAGTGGTGCACCCCCACCACGCCCTGAACAAGATCAG
CAACAGAAGCCTGAGACAGAGACAGATGGACAGACACGCCAAGATCAAG
AGAGCCTACTTCAGCAGCCCTAGCTTCCCCAACTTCTTCAGCACCCTGA
TCAACAGATGCCTGCAGAGAAAGATGACCGGCGAGCCCGACAACAACAG
ATCCACTAGCGTGGAGCTAACCGGCGACCCCAACAAGACAAGAGGCGCC
CCCGAGGCCCTGATGGCCAACAGCGGCGAGCCCTGGAGCCCGAGCTACC
TGGGGCCCACAAGCCCC

The sequences of the present BEs, TM/IC domains, and BE-TM/IC constructs described in section (i) are summarized in Table 1.

TABLE 1
Sequence summary of the present BEs,
TM/IC domains and BE-TM/IC constructs

| | SEQ | |
|---|---|---|
| | ID | |
| Name | NO | Note |
| syn | 1 | Nucleotide sequence of syn CNR2-coding sequence |
| CNR2 | 2 | Amino acid sequence of syn CNR2 |
| syn | 3 | Nucleotide sequence of syn HCAR2-coding |
| HCAR2 | | sequence |
| | 4 | Amino acid sequence of syn HCAR2 |
| syn | 5 | Nucleotide sequence of syn GPR84-coding sequence |
| GPR84 | 6 | Amino acid sequence of syn GPR84 |
| syn | 7 | Nucleotide sequence of syn P2Y14-coding sequence |
| P2Y14 | 8 | Amino acid sequence of syn P2Y14 |
| anti-CD3 | 9 | Nucleotide sequence of anti-CD3 scFv-coding |
| scFv | | sequence |
| | 10 | Amino acid sequence of anti-CD3 scFv |
| anti-FRa | 11 | Nucleotide sequence of anti-FRa scFv-coding |
| scFv | | sequence |
| | 12 | Amino acid sequence of anti-FRa scFv |
| anti-HER2 | 13 | Nucleotide sequence of anti-HER2 scFv-coding |
| scFv | | sequence |
| | 14 | Amino acid sequence of anti-HER2 scFv |
| anti-NKp46 | 25 | Nucleotide sequence of anti-NKp46 scFv-coding |
| scFv | | sequence |
| | 26 | Amino acid sequence of anti-NKp46 scFv |
| anti-CD2 | 52 | Nucleotide sequence of anti-CD2 scFv-coding |
| scFv | | sequence |
| | 53 | Amino acid sequence of anti-CD2 scFv |
| anti-CD2 | 54 | Nucleotide sequence of anti-CD2 scFv-coding |
| scFv | | sequence |
| | 55 | Amino acid sequence of anti-CD2 scFv |
| anti-ICOS | 56 | Nucleotide sequence of anti-ICOS scFv-coding |
| scFv | | sequence |
| | 57 | Amino acid sequence of anti-ICOS scFv |
| ICOS-L | 58 | Nucleotide sequence of ICOS-L-coding sequence |
| peptide | 59 | Amino acid sequence of ICOS-L |
| CAMD1 | 60 | Nucleotide sequence of CAMD1-coding sequence |
| peptide | 61 | Amino acid sequence of CAMD1 |
| IL-2 binder | 62 | Nucleotide sequence of IL-2 binder-coding sequence |
| | 63 | Amino acid sequence of IL-2 binder |
| CD3-syn | 15 | Nucleotide sequence of CD3-syn HCAR2-coding |
| HCAR2 | | sequence |
| | 16 | Amino acid sequence of CD3-syn HCAR2 |

(ii) 2xBE-TM/IC Polypeptides and Nucleic Acids Encoding the Same

For the purpose of eliciting an antigen-targeting effect, the BE-TM/IC polypeptide described in section (i) may further comprise a second BE (for example, an anti-antigen scFv), i.e., in the form of 2xBE-TM/IC domain. Accordingly, the second aspect of the present disclosure pertains to a 2xBE-TM/IC polypeptide, and a nucleic acid encoding the 2xBE-TM/IC polypeptide.

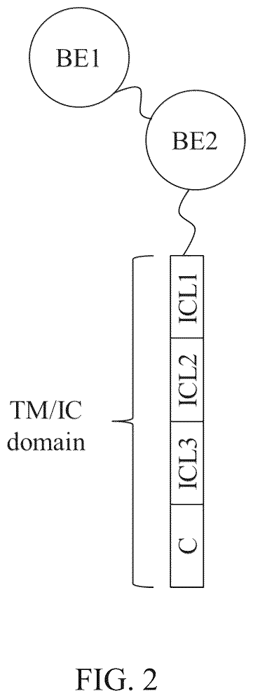

Reference is now made to FIG. 2, which depicts a schematic diagram of the 2xBE-TM/IC polypeptide in accordance with one embodiment of the present disclosure. In structure, the 2xBE-TM/IC polypeptide comprises, a first BE (BE1), a second BE (BE2) linked to the C-terminus of the first BE (BE1), and a TM/IC domain linked to the C-terminus of the second BE (BE2). Preferably, the first BE is an anti-antigen scFv (a first scFv; e.g., an anti-TAA scFv), and the second BE is a cell targeting scFv (a second scFv; e.g., an anti-CD3 scFv, anti-NKp46 scFv, anti-CD2 scFv or anti-ICOS scFv) or a cell targeting peptide (e.g., an ICOS-L, CAMD1 or IL-2 binder). As described in section (i) above, each of the anti-antigen scFv and cell targeting scFv comprises a heavy chain variable domain (VH) and a light chain variable domain (VL), and the TM/IC domain comprises, from its N-terminus to C-terminus, in sequence, the ICL1, ICL2, ICL3 and C-terminal region (C) of a GPCR.

Optionally, the anti-antigen scFv, cell targeting scFv/peptide and TM/IC domain may be linked with or without a linker sequence. Preferably, the anti-antigen scFv and cell targeting scFv/peptide are linked via a first linker sequence, and the cell targeting scFv/peptide and TM/IC domain are linked via a second linker sequence. As would be appreciated, the first and second linker sequences may be the same or different.

According to some preferred embodiments of the present disclosure, the anti-antigen scFv is an anti-TAA scFv. Depending on intended purpose, the TAA may be any antigen over-expressed on or associated by tumor/cancer cells; for example, alphafetoprotein (AFP), carcinoembryonic antigen (CEA), carbohydrate antigen 19-9 (CA 19-9), epidermal growth factor receptor (HER1), HER2, HER3, HER4, epithelial tumor antigen (ETA), folate receptor alpha (FRa), ganglioside GD2, Globo H, melanoma-associated antigen (MAGE), mucin 1 (MUC 1), mucin 16 (MUC 16, also known as “ovarian cancer-related tumor marker CA125”), mesothelin, prostate-specific membrane antigen (PSMA), prostate stem cell antigen (PSCA), receptor tyrosine kinase like orphan receptor 1 (ROR1), glypican 3 (GPC3), tumor-associated glycoprotein 72 (TAG72), or claudin-18 isoform 2 (CLDN18.2). In one embodiment, the anti-antigen scFv is an anti-FRa scFv. According to one example of the present disclosure, the anti-FRa scFv comprises the amino acid sequence of SEQ ID NO: 12. In another embodiment, the anti-antigen scFv is an anti-HER2 scFv. According to one example of the present disclosure, the anti-HER2 scFv comprises the amino acid sequence of SEQ ID NO: 14.

According to certain preferred embodiments, the cell targeting scFv/peptide is specific to a receptor, co-receptor, co-stimulatory molecule, or cell adhesion molecule of an immune cell (e.g., a T cell, NK cell, B cell, basophil, cosinophil, DC or neutrophil); for example, an anti-CD3 scFv, anti-NKp46 scFv, anti-CD2 scFv, anti-ICOS scFv, ICOS-L peptide, CAMD1 peptide, or IL-2 binder. A skilled artisan may choose a suitable scFv/peptide for use in the present invention in accordance with intended purpose, e.g., the immune cell intended to be activated.

As described above, the TM/IC domain is characterized by having the intracellular domain (including ICL1, ICL2, ICL3 and C-terminal region) of the GPCR, without including the extracellular domain and transmembrane domain of the GPCR, in which the ICL1, ICL2, ICL3 and C-terminal region of the TM/IC domain may be linked with or without a linker sequence. Preferably, the present TM/IC domain consists of the ICL1, ICL2, ICL3 and C-terminal region of the GPCR. The GPCR is preferably a class A GPCR; for example, CNR2, HCAR2, GPR84, or P2Y14.

According to some embodiments of the present disclosure, the TM/IC domain may be any of syn CNR2 (SEQ ID NO: 2), syn HCAR2 (SEQ ID NO: 4), syn GPR84 (SEQ ID NO: 6) or syn P2Y14 (SEQ ID NO: 8) as described in section (i) of the present disclosure.

According to some embodiments, the 2BE-TM/IC polypeptide comprises two scFvs linked to the N-terminus of the TM/IC domain, i.e., in the form of 2xscFv-TM/IC polypeptide. According to one exemplary embodiment of the present disclosure, the 2xscFv-TM/IC polypeptide designated as “FRa-CD3-syn HCAR2” comprises, from N-terminus to C-terminus, in sequence, an anti-FRa scFv (e.g., the anti-FRa scFv of SEQ ID NO: 12), an anti-CD3 scFv (e.g., the anti-CD3 scFv of SEQ ID NO: 10), and the syn HCAR2 of the present disclosure. In one exemplary embodiment, the FRa-CD3-syn HCAR2 polypeptide comprises the amino acid sequence of SEQ ID NO: 18.

According to some embodiments, the 2xscFv-TM/IC polypeptide designated as “FRa-CD3-syn CNR2” comprises, from N-terminus to C-terminus, in sequence, an anti-FRa scFv (e.g., the anti-FRa scFv of SEQ ID NO: 12), an anti-CD3 scFv (e.g., the anti-CD3 scFv of SEQ ID NO: 10), and the syn CNR2 of the present disclosure. In one exemplary embodiment, the FRa-CD3-syn CNR2 polypeptide comprises the amino acid sequence of SEQ ID NO: 20.

According to certain embodiments, the 2xscFv-TM/IC polypeptide designated as “FRa-CD3-syn P2Y14” comprises, from N-terminus to C-terminus, in sequence, an anti-FRa scFv (e.g., the anti-FRa scFv of SEQ ID NO: 12), an anti-CD3 scFv (e.g., the anti-CD3 scFv of SEQ ID NO: 10), and the syn P2Y14 of the present disclosure. In one exemplary embodiment, the FRa-CD3-syn P2Y14 polypeptide comprises the amino acid sequence of SEQ ID NO: 22.

According to alternative embodiments, the 2xscFv-TM/IC polypeptide designated as “HER2-CD3-syn GPR84” comprises, from N-terminus to C-terminus, in sequence, an anti-HERs scFv (e.g., the anti-HER2 scFv of SEQ ID NO: 14), an anti-CD3 scFv (e.g., the anti-CD3 scFv of SEQ ID NO: 10), and the syn GPR84 of the present disclosure. In one exemplary embodiment, the HER2-CD3-syn GPR84 polypeptide comprises the amino acid sequence of SEQ ID NO: 24.

Also disclosed herein are nucleic acids encoding the present 2xBE-TM/IC polypeptides. The nucleic acid comprises a promoter, a first coding sequence encoding the first BE (i.e., the anti-antigen scFv; for example, an anti-TAA scFv), a second coding sequence encoding the second BE (i.e., the cell targeting scFv/peptide; for example, a CD3 scFv or anti-NKp46 scFv), and a third coding sequence encoding the present TM/IC domain, in which the first, second and third coding sequences are operably linked to the promoter, the second coding sequence is disposed downstream to the first coding sequence, and the third coding sequence is disposed downstream to the second coding sequence.

As described above, the promoter may be an inducible promoter (e.g., a heat shock inducible promoter, metallothionein promoter, ecdysone-inducible promoter, FKBP dimerization inducible promoter, Gal4-estrogen receptor fusion protein regulated promoter, steroid inducible promoter, streptogramin responsive promoter or tetracycline regulated promoter) or a constitutive promoter (e.g., a CMV promoter, RSV promoter, SV40 promoter, MMTV promoter, PGK promoter, EF1-α promoter, human H1 promoter or U6 promoter). According to one exemplary embodiment, the promoter is EF1-α promoter.

According to some embodiments, the anti-antigen scFv is an anti-FRa scFv, in which the anti-FRa scFv-coding sequence comprises a nucleotide sequence at least 85% identical to SEQ ID NO: 11; preferably, at least 90% identical to SEQ ID NO: 11; more preferably, at least 95% identical to SEQ ID NO: 11. In one specific example, the anti-FRa scFv-coding sequence comprises the nucleotide sequence of SEQ ID NO: 11.

According to certain embodiments, the anti-antigen scFv is an anti-HER2 scFv, in which the anti-HER2 scFv-coding sequence comprises a nucleotide sequence at least 85% identical to SEQ ID NO: 13; preferably, at least 90% identical to SEQ ID NO: 13; more preferably, at least 95% identical to SEQ ID NO: 13. In one specific example, the anti-HER2 scFv-coding sequence comprises the nucleotide sequence of SEQ ID NO: 13.

According to some embodiments, the nucleic acid encodes the FRa-CD3-syn HCAR2 (SEQ ID NO: 18), and comprises a nucleotide sequence at least 85% identical to SEQ ID NO: 17; preferably, at least 90% identical to SEQ ID NO: 17; more preferably, at least 95% identical to SEQ ID NO: 17. According to one exemplary embodiment, the nucleic acid encoding the FRa-CD3-syn HCAR2 comprises the nucleotide sequence of SEQ ID NO: 17.

According to some embodiments, the nucleic acid encodes the FRa-CD3-syn CNR2 (SEQ ID NO: 20), and comprises a nucleotide sequence at least 85% identical to SEQ ID NO: 19; preferably, at least 90% identical to SEQ ID NO: 19; more preferably, at least 95% identical to SEQ ID NO: 19. According to one exemplary embodiment, the nucleic acid encoding the FRa-CD3-syn CNR2 comprises the nucleotide sequence of SEQ ID NO: 19.

According to certain embodiments, the nucleic acid encode the FRa-CD3-syn P2Y14 (SEQ ID NO: 22), and comprises a nucleotide sequence at least 85% identical to SEQ ID NO: 21; preferably, at least 90% identical to SEQ ID NO: 21; more preferably, at least 95% identical to SEQ ID NO: 21. According to one exemplary embodiment, the nucleic acid encoding the FRa-CD3-syn P2Y14 comprises the nucleotide sequence of SEQ ID NO: 21.

According to other embodiments, the nucleic acid encode the HER2-CD3-syn GPR84 (SEQ ID NO: 24), and comprises a nucleotide sequence at least 85% identical to SEQ ID NO: 23; preferably, at least 90% identical to SEQ ID NO: 23; more preferably, at least 95% identical to SEQ ID NO: 23. According to one exemplary embodiment, the nucleic acid encoding the HER2-CD3-syn GPR84 comprises the nucleotide sequence of SEQ ID NO: 23.

The sequences of the present 2xBE-TM/IC constructs described in section (ii) are summarized in Table 2.

TABLE 2
Sequence summary of the present 2xBE-TM/IC constructs

| | SEQ ID | |
|---|---|---|
| Name | NO | Note |
| FRa-CD3-syn | 17 | Nucleotide sequence comprising, from |
| HCAR2 | | 5′ end to 3′ end, in sequence, |
| | | the sequences of SEQ ID NOs: 11, 9 and 3 |
| | 18 | Amino acid sequence comprising, from |
| | | N-terminus to C-terminus, in sequence, |
| | | the sequences of SEQ ID NOs: 12, 10 and 4 |
| FRa-CD3-syn | 19 | Nucleotide sequence comprising, from |
| CNR2 | | 5′ end to 3′ end, in sequence, |
| | | the sequences of SEQ ID NOs: 11, 9 and 1 |
| | 20 | Amino acid sequence comprising, from |
| | | N-terminus to C-terminus, in sequence, |
| | | the sequences of SEQ ID NOs: 12, 10 and 2 |
| FRa-CD3-syn | 21 | Nucleotide sequence comprising, from |
| P2Y14 | | 5′ end to 3′ end, in sequence, |
| | | the sequences of SEQ ID NOs: 11, 9 and 7 |
| | 22 | Amino acid sequence comprising, from |
| | | N-terminus to C-terminus, in sequence, |
| | | the sequences of 12, 10 and 8 |
| HER2-CD3-syn | 23 | Nucleotide sequence comprising, from |
| GPR84 | | 5′ end to 3′ end, in sequence, |
| | | the sequences of SEQ ID NOs: 13, 9 and 5 |
| | 24 | Amino acid sequence comprising, from |
| | | N-terminus to C-terminus, in sequence, |
| | | the sequences of SEQ ID NOs: 14, 10 and 6 |

(iii) 2xBE-multiTM/IC Polypeptides and Nucleic Acids Encoding the Same

In addition to the TM/IC domain described in sections (i) and (ii) above that comprises the ICL1, ICL2, ICL3 and C-terminal region of a single GPCR, the inventor of the present disclosure further discovered that the combination of two or three TM/IC domains may achieve the same bi-functional effect, i.e., the TM/IC domains may act together as a bi-functional domain for anchoring the polypeptide to cell membrane and mediating intracellular signal transduction. Thus, the third aspect of the present pertain to a 2xBE-multiTM/IC polypeptide, and a nucleic acid encoding the 2xBE-multiTM/IC polypeptide.

The configuration of the 2xBE-multiTM/IC polypeptide is quite similar to that of 2xBE-TM/IC polypeptide described in section (ii) above, except the 2xBE-multiTM/IC comprises two or three TM/IC domains acting together as a bi-function domain, rather than one TM/IC domain.

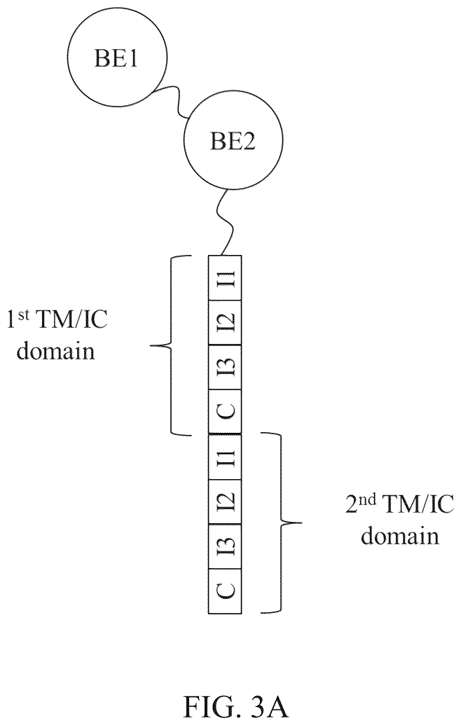

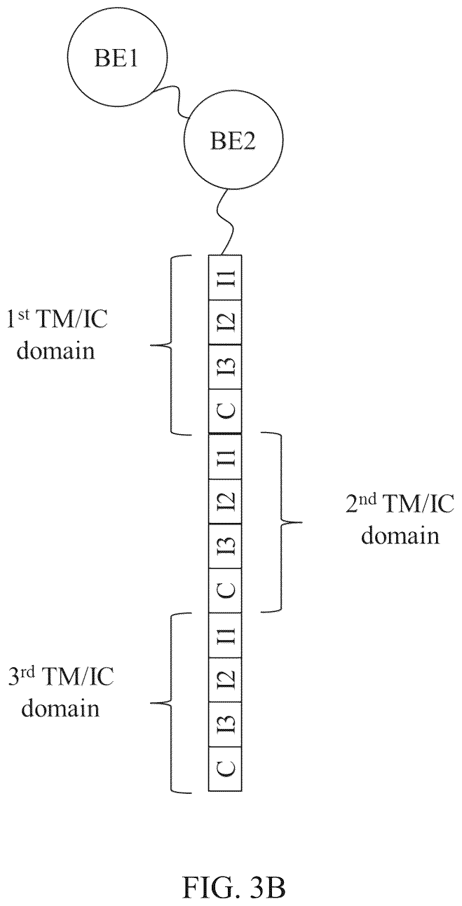

FIG. 3A provides a schematic diagram of the 2xBE-multiTM/IC in accordance with one embodiment of the present disclosure. As depicted in FIG. 3A, the 2xscFv-multiTM/IC comprises two TM/IC domains, in which the first TM/IC domain (1^{st} TM/IC domain) comprises the ICL1 (11), ICL2 (12), ICL3 (13) and C-terminal region (C) of a first GPCR, and the second TM/IC domain (2^{nd} TM/IC domain) comprises the ICL1 (11), ICL2 (12), ICL3 (13) and C-terminal region (C) of a second GPCR. Preferably, each of the first and second GPCRs is a class A GPCR, for example, CNR2, HCAR2, GPR84 and P2Y14; and the second GPCR is different from the first GPCR. Neither the first TM/IC domain nor the second TM/IC domain comprises the extracellular domain and transmembrane domain of the GPCRs (i.e., the first and second GPCRs). As described above, the ICL1, ICL2, ICL3 and C-terminal region of each first and second TM/IC domains may be linked with or without a linker sequence. Preferably, the first TM/IC domain consists of the ICL1, ICL2, ICL3 and C-terminal region of the first GPCR, and the second TM/IC domain consists of the ICL1, ICL2, ICL3 and C-terminal region of the second GPCR.

In certain preferred embodiments, the first and second TM/IC domains are linked to each other without a linker sequence. Alternatively, the first and second TM/IC domains may be linked via a suitable linker sequence.

According to one embodiment, the 2xBE-multiTM/IC polypeptide comprises the syn CNR2 and syn P2Y14 (in the form of 2xTM/IC domain); in this embodiment, the 2xTM/IC domain is designated as “syn CP” and comprises the following amino acid sequence:

(SEQ ID NO: 30)
“SSHQLRRKPSYLDRYLCLRYPPSYKALLTRGRKAHQHVASLSGHQDRQ
VPGMARMRLDVRLAKTRSGEIRSSAHHCLAHWKKCVRGLGSEAKEEAPR
SSVTETEADGKITPWPDSRDLDLSDCPSSKSDRYYKIVKPLWTSFIQSV
SYSKTKKIFKSHLKSSRNSTSVKKKSSRNQPFREILCKKLHIPLKAQND
LDISRIKRGNTTLESTDTL”,

in which the amino acid sequence of syn CNR2 (SEQ ID NO: 2) is marked in italic, and the amino acid sequence of syn P2Y14 (SEQ ID NO: 8) is marked in bold.

According to another embodiment, the 2xBE-multiTM/IC polypeptide comprises the syn CNR2 and syn HCAR2 (in the form of 2xTM/IC domain); in this embodiment, the 2xTM/IC domain is designated as “syn CH” and comprises the following amino acid sequence:

(SEQ ID NO: 32)
“SSHQLRRKPSYLDRYLCLRYPPSYKALLTRGRKAHQHVASLSGHQDRQ
VPGMARMRLDVRLAKTRSGEIRSSAHHCLAHWKKCVRGLGSEAKEEAPR
SSVTETEADGKITPWPDSRDLDLSDCHLKSWKSSRDRYFRVVHPHHALN
KISNRSLRQRQMDRHAKIKRAYFSSPSFPNFFSTLINRCLQRKMTGEPD
NNRSTSVELTGDPNKTRGAPEALMANSGEPWSPSYLGPTSP”,

in which the syn CNR2 (SEQ ID NO: 2) is marked in italic, and the syn HCAR2 (SEQ ID NO: 4) is marked in bold.

FIG. 3B provides a schematic diagram of the 2xBE-multiTM/IC according to another embodiment of the present disclosure. As depicted in FIG. 3B, the 2xBE-multiTM/IC comprises three TM/IC domains, in which the first TM/IC domain (1^{st} TM/IC domain) comprises the ICL1 (11), ICL2 (12), ICL3 (13) and C-terminal region (C) of a first GPCR, the second TM/IC domain (2^{nd} TM/IC domain) comprises the ICL1 (11), ICL2 (12), ICL3 (13) and C-terminal region (C) of a second GPCR, and a third TM/IC domain (3^{rd} TM/IC domain) comprises the ICL1 (11), ICL2 (12), ICL3 (13) and C-terminal region (C) of a third GPCR. Preferably, each of the first, second and third GPCRs is a class A GPCR, for example, CNR2, HCAR2, GPR84 and P2Y14; and the first to the third GPCRs are different from one another. None of the first to the third TM/IC domains comprises the extracellular domain and transmembrane domain of the GPCRs. As described above, the ICL1, ICL2, ICL3 and C-terminal region of each first, second and third TM/IC domains may be linked with or without a linker sequence. Preferably, the first TM/IC domain consists of the ICL1, ICL2, ICL3 and C-terminal region of the first GPCR, the second TM/IC domain consists of the ICL1, ICL2, ICL3 and C-terminal region of the second GPCR, and the third TM/IC domain consists of the ICL1, ICL2, ICL3 and C-terminal region of the third GPCR.

In certain preferred embodiments, the first to the third TM/IC domains are linked to without a linker sequence (i.e., in the form of “1^{st} TM/IC domain-2^{nd} TM/IC domain-3^{rd} TM/IC domain”). Alternatively, the first to the third TM/IC domains may be linked via a suitable linker sequence, e.g., in the form of “1^{st} TM/IC domain-linker-2^{nd} TM/IC domain-linker-3^{rd} TM/IC domain”, “1^{st} TM/IC domain-2^{nd} TM/IC domain-linker-3^{rd} TM/IC domain” or “1^{st} TM/IC domain-linker-2^{nd} TM/IC domain-3^{rd} TM/IC domain”.

According to one embodiment, the 2xBE-multiTM/IC polypeptide comprises the syn CNR2, syn P2Y14 and syn HCAR2 (in the form of 3xTM/IC domain), in which the 3xTM/IC domain is designated as “syn CPH” and comprises the following amino acid sequence:

(SEQ ID NO: 28)
“SSHQLRRKPSYLDRYLCLRYPPSYKALLTRGRKAHQHVASLSGHQDRQ
VPGMARMRLDVRLAKTRSGEIRSSAHHCLAHWKKCVRGLGSEAKEEAPR
SSVTETEADGKITPWPDSRDLDLSDCPSSKSDRYYKIVKPLWTSFIQSV
SYSKTKKIFKSHLKSSRNSTSVKKKSSRNQPFREILCKKLHIPLKAQND
LDISRIKRGNTTLESTDTLHLKSWKSSRDRYFRVVHPHHALNKISNRSL
RQRQMDRHAKIKRAYFSSPSFPNFFSTLINRCLQRKMTGEPDNNRSTSV
ELTGDPNKTRGAPEALMANSGEPWSPSYLGPTSP”,

in which the syn CNR2 sequence (SEQ ID NO: 2) is marked in italic, the syn HCAR2 sequence (SEQ ID NO: 4) is marked in bold, and the syn P2Y14 sequence (SEQ ID NO: 8) is disposed between the syn CNR2 and syn HCAR2 sequences.

Depending on desired purpose, the 2xTM/IC domain or 3xTM/IC domain may be linked to one or two scFvs/peptide (e.g., the anti-antigen scFv and/or cell targeting scFv/peptide) as described above, thereby forming a scFv/peptide-2xTM/IC polypeptide (comprising one scFv/peptide and two TM/IC domains), a 2xscFv/peptide-2xTM/IC polypeptide (comprising two scFvs/peptide and two TM/IC domains), scFv/peptide-3xTM/IC polypeptide (comprising one scFv/peptide and three TM/IC domains), or a 2xscFv/peptide-3xTM/IC polypeptide (comprising two scFvs/peptide and three TM/IC domains).

In one embodiment, a scFv-3xTM/IC polypeptide designated as “HER2-CD3-syn CPH” is provided, which comprises, from N-terminus to C-terminus, in sequence, an anti-HER2 scFv, an anti-CD3 scFv, and the syn CPH. According to the embodiment, the HER2-CD3-syn CPH polypeptide comprises the amino acid sequence of SEQ ID NO: 42.

In another embodiment, a scFv-3xTM/IC polypeptide designated as “FRa-CD3-syn CPH” is provided, which comprises, from N-terminus to C-terminus, in sequence, an anti-FRa scFv, an anti-CD3 scFv, and the syn CPH. According to the embodiment, the FRa-CD3-syn CPH polypeptide comprises the amino acid sequence of SEQ ID NO: 44.

In still another embodiment, a scFv-2xTM/IC polypeptide designated as “FRa-CD3-syn CP” is provided, which comprises, from N-terminus to C-terminus, in sequence, an anti-FRa scFv, an anti-CD3 scFv, and the syn CP. According to the embodiment, the FRa-CD3-syn CP polypeptide comprises the amino acid sequence of SEQ ID NO: 48.

In a further embodiment, a scFv-2xTM/IC polypeptide designated as “FRa-CD3-syn CH” is provided, which comprises, from N-terminus to C-terminus, in sequence, an anti-FRa scFv, an anti-CD3 scFv, and the syn CH. According to the embodiment, the FRa-CD3-syn CH polypeptide comprises the amino acid sequence of SEQ ID NO: 50.

The present disclosure also provides nucleic acids encoding the 2xBE-multiTM/IC polypeptides. According to certain embodiments of the present disclosure, the nucleic acid comprises a promoter, a first coding sequence encoding the first BE (i.e., an anti-antigen scFv; for example, an anti-TAA scFv), a second coding sequence encoding the second BE (i.e., a cell targeting scFv/peptide; for example, an anti-CD3 scFv or anti-NKp46 scFv), and a third coding sequence encoding the multiTM/IC domain, in which the first, second and third coding sequences are operably linked to the promoter, the second coding sequence is disposed downstream to the first coding sequence, and the third coding sequence is disposed downstream to the second coding sequence.

The promoter, first coding sequence and second coding sequence are quite similar to that of 2xBE-TM/IC construct described in section (ii) of the present disclosure. Hence, the detain description thereof is omitted for the sake of brevity.

The difference of the third coding sequence of 2xBE-multiTM/IC construct and the third coding sequence of 2xBE-TM/IC construct lies in that the third coding sequence of 2xBE-multiTM/IC comprises multiple (two or three) coding segments for expressing multiple (two or three) TM/IC domains; for example, the third coding sequence may comprise two coding segments respectively expressing the syn CNR2 and syn P2T14 (i.e., the syn CP), or respectively expressing the syn CNR2 and syn HCAR2 (i.e., the syn CH); alternatively, the third coding sequence may comprise three coding segments respectively expressing the syn CNR2, syn P2T14 and syn HCAR2 (i.e., the syn CPH).

According to some embodiments, the third coding sequence encodes the syn CP (SEQ ID NO: 30), and comprises a nucleotide sequence at least 85% identical to SEQ ID NO: 29; preferably, 90% identical to SEQ ID NO: 29; more preferably, 100% identical to SEQ ID NO: 29; most preferably, 100% identical to SEQ ID NO: 29. According to some embodiments, the third coding sequence encodes the syn CH (SEQ ID NO: 32), and comprises a nucleotide sequence at least 85% identical to SEQ ID NO: 31; preferably, 90% identical to SEQ ID NO: 31; more preferably, 100% identical to SEQ ID NO: 31; most preferably, 100% identical to SEQ ID NO: 31. According to other embodiments, the third coding sequence encodes the syn CPH (SEQ ID NO: 28), and comprises a nucleotide sequence at least 85% identical to SEQ ID NO: 27; preferably, 90% identical to SEQ ID NO: 27; more preferably, 100% identical to SEQ ID NO: 27; most preferably, 100% identical to of SEQ ID NO: 27.

In one working example, the nucleic acid encoding the HER2-CD3-syn CPH polypeptide (SEQ ID NO: 42) comprises the nucleotide sequence of SEQ ID NO: 41. In another working example, the nucleic acid encoding the FRa-CD3-syn CPH polypeptide (SEQ ID NO: 44) comprises the nucleotide sequence of SEQ ID NO: 43. In another working example, the nucleic acid encoding the FRa-CD3-syn CP polypeptide (SEQ ID NO: 48) comprises the nucleotide sequence of SEQ ID NO: 47. In one specific example, the nucleic acid encoding the FRa-CD3-syn CH polypeptide (SEQ ID NO: 50) comprises the nucleotide sequence of SEQ ID NO: 49.

The sequences of the present 2xTM/IC domains, 3xTM/ID domains, 2xBE-2xTM/IC constructs and 2xBE-3xTM/IC constructs described in section (iii) are summarized in Table 3.

TABLE 3
Sequence summary of the present 2xTM/IC domains, 2xBE-
2xTM/IC constructs and 2xBE-3xTM/IC constructs

| | SEQ ID | |
|---|---|---|
| Name | NO | Note |
| syn CP | 29 | Nucleotide sequence |
| | | (SEQ ID NO: 1 + SEQ ID NO: 7) |
| | 30 | Amino acid sequence |
| | | (SEQ ID NO: 2 + SEQ ID NO: 8) |
| syn CH | 31 | Nucleotide sequence |
| | | (SEQ ID NO: 1 + SEQ ID NO: 3) |
| | 32 | Amino acid sequence |
| | | (SEQ ID NO: 2 + SEQ ID NO: 4) |
| syn CPH | 27 | Nucleotide sequence |
| | | (SEQ ID NO: 1 + SEQ ID NO: 7 + |
| | | SEQ ID NO: 3) |
| | 28 | Amino acid sequence |
| | | (SEQ ID NO: 2 + SEQ ID NO: 8 + |
| | | SEQ ID NO: 4) |
| HER2-CD3-syn | 41 | Nucleotide sequence comprising, from |
| CPH | | 5′ end to 3′ end, in sequence, |
| | | the sequences of SEQ ID NOs: 13, 9 and 27 |
| | 42 | Amino acid sequence comprising, from |
| | | N-terminus to C-terminus, in sequence, |
| | | the sequences of SEQ ID NOs: 14, 10 and 28 |
| FRa-CD3-syn | 43 | Nucleotide sequence comprising, from |
| CPH | | 5′ end to 3′ end, in sequence, |
| | | the sequences of SEQ ID NOs: 11, 9 and 27 |
| | 44 | Amino acid sequence comprising, from |
| | | N-terminus to C-terminus, in sequence, |
| | | the sequences of SEQ ID NOs: 12, 10 and 28 |
| FRa-CD3-syn | 47 | Nucleotide sequence comprising, from |
| CP | | 5′ end to 3′ end, in sequence, |
| | | the sequences of SEQ ID NOs: 11, 9 and 29 |
| | 48 | Amino acid sequence comprising, from |
| | | N-terminus to C-terminus, in sequence, |
| | | the sequences of SEQ ID NOs: 12, 10 and 30 |
| FRa-CD3-syn | 49 | Nucleotide sequence comprising, from |
| CH | | 5′ end to 3′ end, in sequence, |
| | | the sequences of SEQ ID NOs: 11, 9 and 31 |
| | 50 | Amino acid sequence comprising, from |
| | | N-terminus to C-terminus, in sequence, |
| | | the sequences of SEQ ID NOs: 12, 10 and 32 |

### (iv) Immune Cells Expressing the Recombinant Polypeptide and Uses Thereof in Treating Cancers

The fourth aspect of the present disclosure is directed to an engineered immune cell having the present recombinant polypeptide (i.e., any of the BE-TM/IC polypeptide, 2xBE-TM/IC polypeptide and 2xBE-multiTM/IC polypeptide as respectively described in sections (i)-(iii) of the present disclosure) expressed thereon.

According to certain embodiments, the present engineered immune cell is prepared by introducing the nucleic acid of the present disclosure into an immune cell via a suitable method, followed by culturing the introduced immune cell under a suitable condition (e.g., 37° C.) for a period of time (e.g., 48-72 hours or longer), thereby allowing the introduced immune cell to express the present polypeptide on its surface. The method of introducing a nucleic acid into a cell for the expression purpose is known by a person having an ordinary skill in the art, for example, a viral method or a non-viral method. Specifically, in the viral method, the nucleic acid encoding the protein of interest (e.g., the nucleic acid encoding the present BE-TM/IC polypeptide, 2xBE-TM/IC polypeptide or 2xBE-multiTM/IC polypeptide) is introduced into the immune cell via a viral vector; exemplary viral vectors suitable for this purpose include, but are not limited to, sendai virus, adenovirus, adeno-associated virus (AAV), retrovirus, lentivirus, and sindbis virus. In the non-viral method, the nucleic acid encoding the protein of interest is introduced into the immune cell via a non-viral technique, such as mRNA transfection, miRNA transfection or infection, transposons (e.g., PiggyBac and Sleeping Beauty), plasmid transfection, liposomal magnetofection, minicircle transfection, electroporation or other methods known to introduce a nucleic acid into a host cell.

The immune cell, according to its intended purpose, may be any of a T cell, NK cell, B cell, basophil, eosinophil, DC or neutrophil. As could be appreciated, the cell targeting scFv of the present recombinant polypeptide would vary with the type of the immune cell. For example, in the case when the immune cell is the T cell, then the cell targeting scFv is an anti-CD3 scFv. Alternatively, in the case when the immune cell is the NK cell, then the cell targeting scFv is an anti-NKp46 scFv.

According to certain embodiments of the present disclosure, the engineered immune cell having the anti-TAA scFv and cell targeting scFv/peptide (e.g., anti-CD3 scFv, anti-NKp46 scFv, anti-CD2 scFv anti-ICOS scFv, ICOS-L peptide, CAMD1 peptide, or IL-2 binder) expressed thereon is capable of targeting and killing TAA-expressing cancers.

In some exemplary embodiment, the engineered immune cell is an engineered T cell. Compared to the engineering T cells with chimeric antigen receptor (CAR-T) or T cell antigen coupler (TAC-T), the engineered immune cell of the present disclosure is characterized by not having any components of, (1) CAR-T co-stimulatory domain (such as CD28 and 4-1BB), (2) CAR-T activation domain (such as CD3-zeta ITAM), (3) CAR-T or TAC-T transmembrane domain, (such as the transmembrane domain of CD28, CD8 or CD4), and (4) CD4 or CD8 intracellular domain to deliver signal to CD3 complex like TAC-T does.

Another aspect of the present disclosure provides a method of treating cancer in a subject. The method comprises administering to the subject an effective amount of the present engineered immune cell.

According to certain exemplary embodiments of the present disclosure, the effective amount of the present engineered immune cell is about 1×10⁵ to 1×10¹⁰ cells per dose, such as 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, or 1×10¹⁰ cells dose.

Depending on intended purpose, the immune cell and the engineered immune cell derived therefrom may be derived from the subject being treated/administered (i.e., autologous transplantation), another subject of the same species (i.e., allogeneic transplantation), or a subject of different species (i.e., xenogeneic transplantation). Preferably, said transplantation is autologous transplantation or allogeneic transplantation. In the case when the transplantation is allogeneic transplantation, the method may further comprise the step of administering to the subject an immunosuppressive treatment prior to, concurrently with, or after the administration of present engineered immune cell, so as to suppress the immune response of the subject against the allogeneic engineered immune cell. The immunosuppression may be achieved by any agent and/or method known by a skilled artisan to prevent graft rejection, for example, the administration of gamma irradiation or immunosuppressant.

Non-limiting examples of the cancer treatable with the present immune cell and/or method include, breast cancer, gastric cancer, colorectal cancer, gallbladder cancer, prostate cancer, cervical cancer, ovarian carcinoma, chronic or acute lymphocytic leukemia, bladder cancer, renal cancer, hepatocellular carcinoma, head and neck squamous cell carcinoma, glioblastoma, esophageal cancer, pancreatic cancer, oral cancer, lung cancer, melanoma, and lymphoma.

The present immune cell may be administered to the subject via a suitable route, such as intratumoral, intravenous, intraarterial or intraperitoneal injection.

Among all embodiments of the present disclosure, the subject is a mammal, for example, a human, mouse, rat, rabbit or monkey. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Construction of Nucleic Acids

In the present disclosure, one scFv-TM/IC construct (referred to as “CD3-syn HCAR2 construct”), four 2xscFv-TM/IC constructs (respectively referred to as “FRa-CD3-syn HCAR2 construct”, “FRa-CD3-syn CNR2 construct”, “HER2-CD3-syn GPR84 construct” and “FRa-CD3-syn P2Y14 construct”), and four 2xscFv-multiTM/IC constructs (respectively referred to as “HER2-CD3-syn CPH construct”, “FRa-CD3-syn CPH construct”, “FRa-CD3-syn CP construct” and “FRa-CD3-syn CH construct”) were provided.

In the preparation of the scFv-TM/IC construct, each scFv DNA sequence, including the selected TM/IC DNA sequence, was optimized according to human DNA codon usage. The scFv-TM/IC DNA sequence was entirely designed de novo and subsequently in vitro. Using a desired restriction enzyme, the 3^{rd} generation Lentivirus backbone was digested to serve as the vector. Synthesized DNA was amplified by polymerase chain reaction (PCR) to create inserts, which were assembled into the vector using Gibson Assembly® method. DNA was transformed into Stb13 competent Escherichia coli, cultured at 30° C., and the resulting plasmid was verified using Sanger sequencing.

In the preparation of the 2xscFv-TM/IC constructs, two selected scFv sequences were cloned into a 2xscFv construct through de novo linkage, employing optimized DNA codons based on human DNA codon usage. The de novo designed 2xscFv construct was subsequently synthesized in vitro. A desired restriction enzyme was employed to selectively cleave the desired scFv-TM/IC Lentivirus backbone construct, removing the scFv region to serve as the vector. The 2xscFv insert DNA was PCR-amplified from synthetic DNA as the insert. The resulting PCR-amplified inserts were then assembled into the vector utilizing Gibson Assembly® method. DNA was transformed into Stb13 competent Escherichia coli, cultured at 30° C., and the resulting plasmid was verified using Sanger sequencing.

For the purpose of preparing the 2xscFv-multiTM/IC constructs, selected TM/IC sequences were cloned into a multiTM/IC construct through de novo linkage, incorporating optimized DNA codons based on human DNA codon usage. The de novo designed multiTM/IC construct was subsequently synthesized in vitro. Utilizing a desired restriction enzyme, the 3rd generation Lentivirus backbone was selectively cleaved to serve as the vector. The 2xscFv insert DNA was amplified through PCR from the 2xscFV-TM/IC construct, and the multiTM/IC DNA was similarly amplified by PCR. The resulting PCR-amplified inserts were then assembled into the vector utilizing Gibson Assembly® method. DNA was transformed into Stb13 competent Escherichia coli, cultured at 30° C., and the resulting plasmid was verified using Sanger sequencing.

### Cell Culture

The human T cell lymphoblastic lymphoma cell line SupT1 was cultured in RPMI-1640 basal medium supplemented with 10% fetal bovine serum (FBS) and 1× penicillin-streptomycin. Human primary T cells were cultured in RPMI-1640 basal medium supplemented with 10% fetal bovine serum (FBS), 1× beta-mercaptoethanol, 1× sodium pyruvate, and 1× non-essential amino acids (NEAA). The cells were cultured at 37° C. in 5% CO₂ humidified incubator.

The human high-grade serous ovarian adenocarcinoma cell line Ovcar3 or Ovcar4 was cultured in RPMI-1640 basal medium supplemented with 20% (Ovcar3) or 10% (Ovcar4) FBS and 1× penicillin-streptomycin. The cells were cultured at 37° C. in 5% CO₂ humidified incubator.

### Preparation of Lentivirus

The scFv-TM/IC, 2xscFv-TM/IC and 2xscFv-multiTM/IC constructs were respectively made as lentivirus particles and transfected into human SupT1 cells and human primary T cells. In brief, 12 million 293T cells (ATCC, CRL-3216) were plated in a 15-cm plate. One night later, the packaging plasmids (including the plasmids respectively encoding Rev responsive element (RRE; 9.375 ug), REV (9.375 ug), and vesicular stomatitis virus glycoprotein (VSV-G; 3.75 ug)) and the lentiviral backbone plasmid (i.e., the scFv-TM/IC, 2xscFv-TM/IC or 2xscFv-multiTM/IC construct; 7.5 ug) were co-transfected into the 293T cells by lipofectaminex 3000 following the manufacturer's protocol. Supernatant was harvested after 48 hours and filtered with a 0.45 um filter. The thus-obtained product was concentrated, and the lentivirus titer was assessed by evaluating the transfection efficiency in SupT1 cells. Briefly, lentivirus particles were added to SupT1 cells at a concentration of 20,000 cells per well, following a 3-fold serial dilution protocol from a 1:27 dilution to a 1:2187 dilution. Three days after the viral introduction, the expression of Protein L was examined. The cells were incubated in a 37° C. environment with 5% CO₂.

### Preparation of Engineered T Cells

Human primary T cells were cultured in RPMI-1640 basal medium supplemented with 10% FBS, 1× beta-mercaptoethanol, 1× NEAA, 1× Glutamax™, 1× sodium pyruvate, and 10 mM N-2-hydroxyethylpiperazine-N-2-ethane sulfonic acid (HEPES). Human primary T cells were activated by using Dynabeads™ Human T-Activator CD3/CD28 on the day of thawing (day 0). On day 1, viruses (MOI=1-7.5 based on SupT1 titer) were added with Lenti-Boost™ according to the manufacturer's protocol. Protein L expression was examined on day 7, and the cells were harvested on day 9, followed by culturing in a 37° C. incubator with 5% CO₂ through entire process, and then storing in liquid nitrogen.

In the present study, three engineered T cells were used as control groups, including
(a) FRa_CAR-T, an engineered T cell expressing a FRa chimeric antigen receptor (FRa_CAR), which, in its structure, comprised an anti-FRa scFv, a hinge domain, a transmembrane domain, a CD28 co-stimulatory domain, and a CD3 zata ITAM motif (from N-terminus to C-terminus); the FRa chimeric antigen receptor comprised the amino acid sequence of SEQ ID NO: 34, and was encoded by the nucleotide sequence of SEQ ID NO: 33;
(b) HER2_TAC-T, an engineered T cell expressing a TAC comprising an anti-HER2 scFv, an anti-CD3 scFv and a CD4 intracellular domain, in which the TAC comprised the amino acid sequence of SEQ ID NO: 40, and was encoded the nucleotide sequence of SEQ ID NO: 39; and
(c) FRa-TAC-T, an engineered T cell expressing a TAC comprising an anti-FRa scFv, an anti-CD3 scFv and a CD4 intracellular domain, in which the TAC comprised the amino acid sequence of SEQ ID NO: 46, and was encoded by the nucleotide sequence of SEQ ID NO: 45.

### Flow Cytometry

The expression levels of the present scFv-TM/IC, 2xscFv-TM/IC, and 2xscFv-multiTM/IC polypeptides on cell membrane of human SupT1 cells and primary T cells were determined by flow cytometry. In brief, engineered SupT1/primary human cells were washed in phosphate buffered saline (PBS) twice, then stained with viability dye at 4° C. for 30 minutes. Subsequently, the cells were washed in PBS twice and then stained with phycoerythrin (PE)-conjugated protein L antibody at 4° C. for 45 minutes. Cells were then washed twice with PBS and resuspended in 200 μl PBS before being evaluated by flow cytometer. Data were analyzed using software. Surface expression of constructs was determined by gating PE-positive populations within the live cell gate.

### Animal Study

NCG mice were used in the study to evaluate the anti-tumor effect of the present engineered T cell. In brief, 5×10⁶ OVCAR-3 cells in 100 μl PBS were inoculated subcutaneously into the flank of each NCG mice. Tumor size was recorded twice per week. Volumes of subcutaneously xenografted tumors in vivo were determined using a digital caliper, where tumor volume was calculated by the modified ellipsoid formula, ½×(length×width×width). When the average size of tumor reached about 100 mm³, the engineered T cells (0.3×10⁶ or 1.5×10⁶) respectively expressing FRa_CD3_syn CPH and FRa-TAC were intravenously (i.v.) administered to the mice.

### Example 1 Characterization of the Present Engineered T Cells

1.1 Expression of the Present scFv-TM/IC, 2xscFv-TM/IC and 2xscFv-multiTM/IC Constructs

The expressions of the present scFv-TM/IC construct (i.e., CD3-syn HCAR2 construct), 2xscFv-TM/IC constructs (including FRa-CD3-syn HCAR2, FRa-CD3-syn CNR2, HER2-CD3-syn GPR84, and FRa-CD3-syn P2Y14 constructs), and 2xscFv-multiTM/IC constructs (including HER2-CD3-syn CPH, FRa-CD3-syn CPH, FRa-CD3-syn CP, and FRa-CD3-syn CH constructs) on engineered T cells were examined in this example. As described in “Materials and methods” of the present disclosure, human T cell lymphoblastic lymphoma cell line SupT1 and human primary T cells were respectively transfected with the lentivirus expressing the present scFv-TM/IC, 2xscFv-TM/IC and 2xscFc-multiTM/IC polypeptides. After culturing at 37° C. for 7 days, the expression of the present polypeptides was determined by flow cytometry. The data was summarized in Table 4.

TABLE 4
Expression levels of specified constructs on cell
membrane of human SupT1 cells and primary T cells

| SupT1 cell line | | Primary T cells | |
|---|---|---|---|
| | Percentage | | Percentage |
| | of positive | | of positive |
| Construct | cells | Construct | cells |
| CD3-syn HCAR2 | 45.8 | CD3-syn HCAR2 | 84.8 |
| FRa-CD3-syn HCAR2 | 50.4 | FRa-CD3-syn HCAR2 | 89.9 |
| FRa-CD3-syn CNR2 | 79.4 | FRa-CD3-syn CNR2 | 87.3 |
| FRa-CD3-syn P2Y14 | 72.5 | FRa-CD3-syn P2Y14 | 96.1 |
| HER2-CD3-syn | 73.4 | HER2-CD3-syn | 58.6 |
| GPR84 | | GPR84 | |
| HER2-CD3-syn CPH | 80.7 | HER2-CD3-syn CPH | 57.2 |
| FRa-CD3-syn CPH | 99 | FRa-CD3-syn CPH | 99.5 |
| FRa-CD3-syn CP | 96.5 | FRa-CD3-syn CP | 94 |
| FRa-CD3-syn CH | 99.4 | FRa-CD3-syn CH | 92.7 |

The data of Table 4 demonstrated the expression of the present scFv-TM/IC, 2xscFv-TM/IC, and 2xscFv-multiTM/IC polypeptides on cell membrane of human SupT1 cells and primary T cells.

The anti-CD3 scFv in FRa-CD3-syn CPH was further replaced by anti-CD2 scFv (SEQ ID NO: 53), anti-CD2 scFv (SEQ ID NO: 55), anti-ICOS scFv (SEQ ID NO: 57), anti-ICOS-L (SEQ ID NO: 59), CAMD1 (SEQ ID NO: 61), or IL-2 binder (SEQ ID NO: 63). The thus-produced constructs respectively designated as “FRa-CD2 (1)-syn CPH”, “FRa-CD2 (2)-syn CPH”, “FRa-ICOS-syn CPH”, “FRa-ICOS-L-syn CPH”, “FRa-CAMD1-syn CPH”, and “FRa-IL-2 binder-syn CPH” were made as lentivirus particles and then transfected into human primary T cells. After culturing at 37° C. for 7 days, the expression of the present polypeptides was determined by flow cytometry. The data was summarized in Table 5.

TABLE 5
Expression levels of specified constructs
on cell membrane of human primary T cells

| Construct | Percentage of positive cells |
|---|---|
| FRa-CD2(1)-syn CPH | 83.3 |
| FRa-CD2(2)-syn CPH | 87.4 |
| FRa-ICOS-syn CPH | 83.4 |
| FRa-ICOS-L-syn CPH | 52.8 |
| FRa-CAMD1-syn CPH | 66.3 |
| FRa-IL-2 binder-syn CPH | 91.0 |

The data of Table 5 confirmed the membrane expression of the present constructs (in the form of 2xscFv-multiTM/IC or scFv-peptide-multiTM/IC) on T cells.

The data of this example demonstrated that each of the present TM/IC, 2xTM/IC and 3xTM/IC domains is suitable for use as a transmembrane domain for anchoring one or more targeting elements (e.g., scFv(s) and/or peptide(s)) on the surface of immune cells (e.g., T cells).

### 1.2 Effect of CD3-Mediated Pathway on Tumor Killing Activity of Engineered T Cells

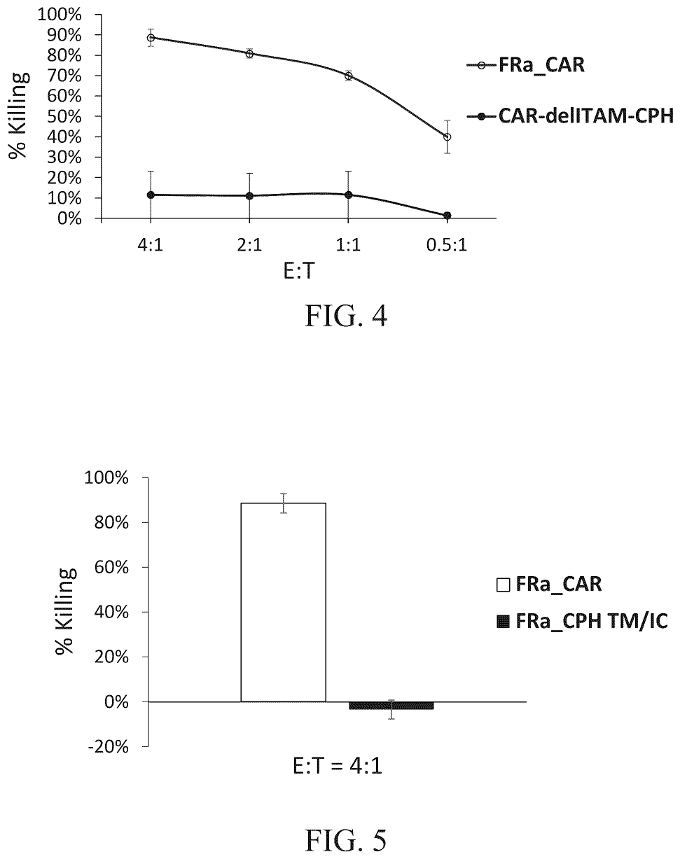

In the example, the ITAM domain of FRa_CAR was replaced with the present syn CPH; the thus-produced polypeptide was designated as “CAR-delITAM-CPH”, which comprised, in the order from N-terminus to C-terminus, an anti-FRa scFv, a hinge domain, a transmembrane domain, a CD28 co-stimulatory domain, and the present syn CPH. According to the sequencing results, the CAR-delITAM-CPH (encoded by the nucleotide sequence of SEQ ID NO: 35) comprised the amino acid sequence of SEQ ID NO: 36. The data of flow cytometry indicated that both the FRa_CAR and CAR-delITAM-CPH were expressed on human primary T cells (data not shown); however, the engineered T cells expressing the CAR-delITAM-CPH exhibited no killing effect on Ovcar4 cancer cells (FIG. 4).

Then, the present syn CPH was linked to an anti-FRa scFv. The thus-produced FRa-CPHTM/IC comprised the amino acid sequence of SEQ ID NO: 38, which was encoded by the nucleotide sequence of SEQ ID NO: 37. According to the results of flow cytometry, the FRa-CPH™/IC could be expressed on human primary T cells (data not shown). Nonetheless, the engineered T cells expressing the FRa-CPHTM/IC exhibited no killing effect on Ovcar4 cancer cells as compared to the FRa_CAR-engineered T cells (FIG. 5).

The data of FIGS. 4 and 5 suggested that the CD3-mediated signal pathway plays an important role in T cell activation and the subsequent tumor killing activity.

### 1.3 Tumor Killing Activity of Engineered T Cells

The tumor killing activity of two exemplary HER2-expressing engineered T cells, including the engineered T cells expressing HER2-CD3-syn GPR84 (HER2-CD3-syn GPR84-T cells) and the engineered T cells expressing HER2-CD3-syn CPH (HER2-CD3-syn CPH-T cells), towards Ovcar4 cancer cells was examined in the example. The HER2_TAC-T served as a control group in the study. According to the data of Table 6, the polypeptide expressed by each of the HER2-CD3 based constructs could be detected on the surface of human primary T cells.

TABLE 6
Expression levels of specified HER2-CD3 based constructs
on cell membrane of human primary T cells

| Construct | Percentage of positive cells |
|---|---|
| Untransduced T cells | 0.73 |
| HER2_TAC-T | 56.9 |
| HER2-CD3-syn CPH | 70.2 |
| HER2-CD3-syn GPR84 | 70.8 |

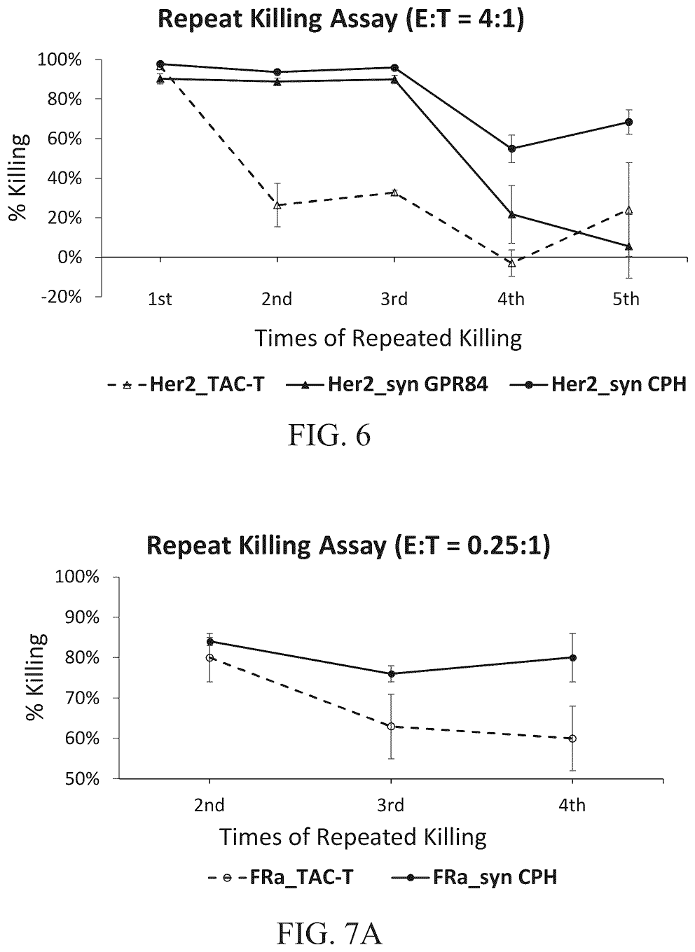

In tumor killing assay, the engineered T cells expressing the HER2-CD3 based constructs were co-cultured with Ovcar4 ovarian cancer cells at an effector to target (E:T) ratio of 4:1, 2:1, 1:1, or 1:2. 48 hours after, the T cells were collected and rest of cancer cells in the well were evaluated viability as a killing result. Meanwhile, T cells were transferred to another well of seeded cancer cells as repeat killing procedure. The data of FIG. 6 (a killing result in total five times repeat) and Table 7 demonstrated that the present engineered T cells exhibited a repeatedly cytotoxic effect on ovarian cancer cells in a long-term killing assay.

TABLE 7
Cytotoxic effect of specified engineered T cells on cancer cells

| Round of killing assay | E:T | HER2-CD3-syn | |
|---|---|---|---|
| (% killing) | ratio | CPH | HER2_TAC-T |
| 1 | 4:1 | 88 | 92 |
| | 2:1 | 93 | 86 |
| | 1:1 | 90 | 80 |
| | 1:2 | 82 | 70 |
| 2 | 4:1 | 99 | 91 |
| | 2:1 | 97 | 64 |
| | 1:1 | 92 | 50 |
| | 1:2 | 78 | 48 |
| 3 | 4:1 | 97 | 38 |
| | 2:1 | 73 | 34 |
| | 1:1 | 59 | 35 |
| | 1:2 | 56 | 33 |
| 4 | 4:1 | 87 | −57 |
| | 2:1 | 4 | −28 |
| | 1:1 | −38 | −27 |
| | 1:2 | −24 | −23 |
| 5 | 4:1 | 73 | 23 |
| | 2:1 | 45 | 5 |
| | 1:1 | 39 | 26 |
| | 1:2 | 29 | 34 |

The effect of the engineered T cells expressing the FRa_CD3_syn CPH (SEQ ID NO: 44; i.e., FRa_CD3_syn CPH-T cells) on Ovcar4 cancer cells was also determined. The FRa-TAC-T and FRa_CAR-T served as a control group in the study. According to the data of Table 8, the polypeptide expressed by each of the FRa based constructs could be detected on the surface of human primary T cells.

TABLE 8
Expression levels of specified FRa based constructs
on cell membrane of human primary T cells

| Construct | Percentage of positive cells |
|---|---|
| Untransduced T cells | 0.72 |
| FRa_CD3_syn CPH-T | 87.1 |
| FRa-TAC-T | 98.1 |
| FRa_CAR-T | 89.3 |

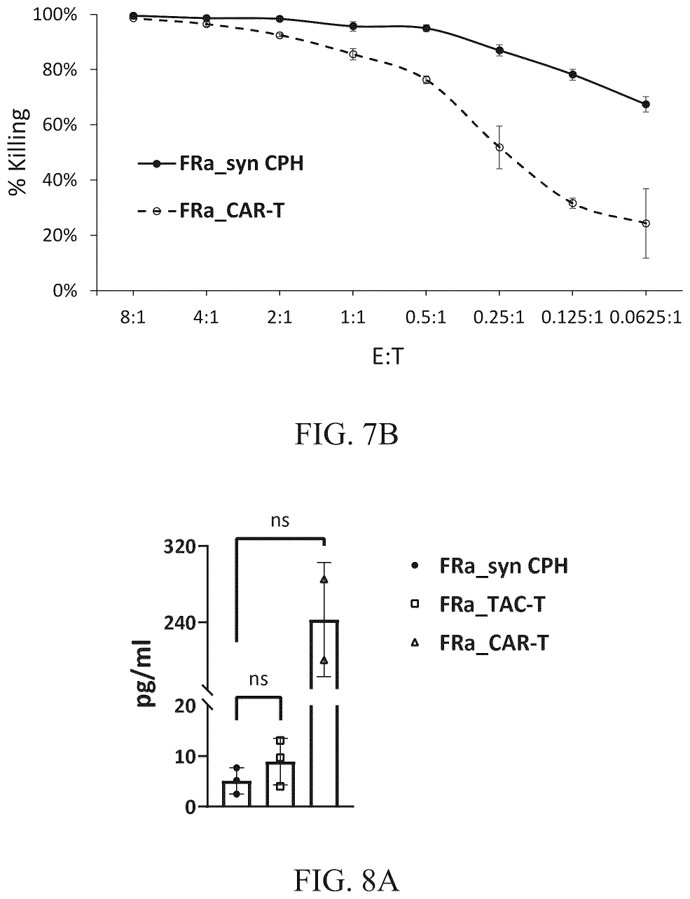

To perform the tumor killing assay, the engineered T cells expressing the FRa based constructs were co-cultured with Ovcar4 ovarian cancer cells at different E:T ratio. 48 hours after, the T cells were collected and rest of cancer cells in the well were evaluated viability as a killing result. Meanwhile, T cells were transferred to another well of seeded cancer cells as repeat killing procedure. The data of FIGS. 7A, 7B, and Table 9 demonstrated the cytotoxic effect of the present engineered T cells on ovarian cancer cells at the E:T ratio ranging from 0.0625:1 to 8:1 in a long-term killing assay.

TABLE 9
Cytotoxic effect of specified engineered T cells in cancer cells

| Round of | | | | |
|---|---|---|---|---|
| killing assay | E:T | FRa_CD3_syn | | |
| (% killing) | ratio | CPH-T | FRa-TAC-T | FRa_CAR-T |
| 1 | 4:1 | 96 | 96 | 89 |
| | 2:1 | 95 | 94 | 81 |
| | 1:1 | 95 | 86 | 70 |
| | 0.5:1 | 92 | 70 | 40 |
| 2 | 4:1 | 98 | 97 | 94 |
| | 2:1 | 99 | 95 | 87 |
| | 1:1 | 98 | 87 | 85 |
| | 0.5:1 | 96 | 77 | 47 |
| 3 | 4:1 | 76 | 73 | 77 |
| | 2:1 | 76 | 72 | 69 |
| | 1:1 | 76 | 62 | 60 |
| | 0.5:1 | 70 | 61 | 18 |
| 4 | 4:1 | 82 | 74 | 85 |
| | 2:1 | 87 | 71 | 65 |
| | 1:1 | 76 | 61 | 54 |
| | 0.5:1 | 72 | 56 | 40 |
| 5 | 4:1 | 78 | 56 | 78 |
| | 2:1 | 79 | 55 | 50 |
| | 1:1 | 72 | 42 | 34 |
| | 0.5:1 | 54 | 30 | 17 |

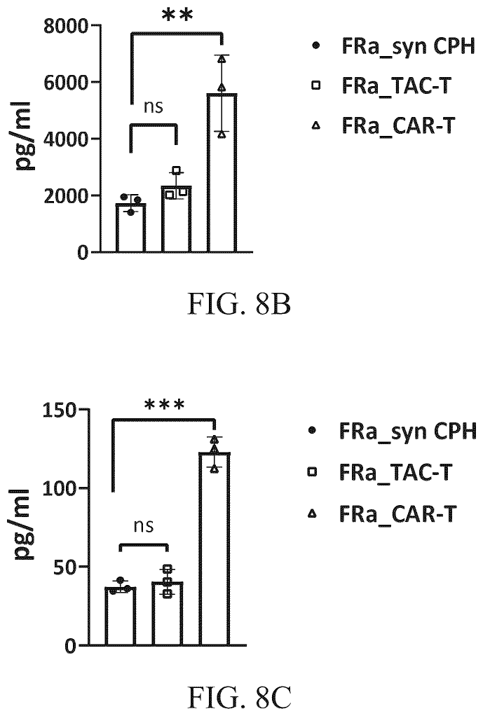

In addition to Ovcar4 cancer cells, the anti-tumor effect of the engineered T cells expressing the FRa_CD3_syn CPH (i.e., FRa_CD3_syn CPH-T cells) on OVCAR-3 cancer cells, a high-grade serous ovarian adenocarcinoma cell line isolated from the malignant ascites of an ovary patient, was also determined in this example. The FRa-TAC-T and FRa_CAR-T served as a control group in the study. The engineered T cells were co-cultured with OVCAR-3 cancer cells at an E:T ratio of 1:1. 72 hours after, the T cells were transferred to another well of seeded cancer cells as repeat killing procedure. After the 2^{nd} round of killing assay, the supernatants were collected and subjected to cytokine assays. The percentages of viable cells (%) treated with the present FRa_CD3_syn CPH-T, FRa-TAC-T and FRa_CAR-T were about 1% (standard error: 0%), 2% (standard error: 1%) and 2% (standard error: 0%), respectively (data not shown), demonstrating the tumor-killing activities of the tested FRa-targeting engineered T cells. According to the results of cytokine assays, compared to the positive controls (i.e., FRa-TAC-T and FRa_CAR-T), the treatment of the present FRa_CD3_syn CPH-T cells induced obvious higher expression levels of pro-inflammatory Th1 cytokines, including IL-2 (FIG. 8A), IFN-γ (FIG. 8B), and TNF-α (FIG. 8C).

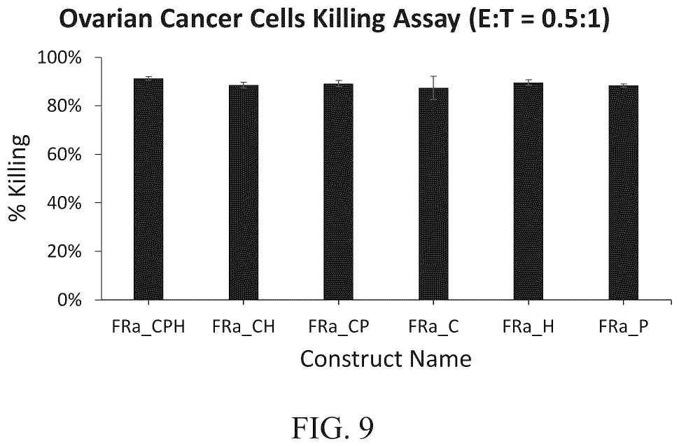

On the other hand, the data of FIG. 9 confirmed that each of the engineered T cells respectively expressing FRa-CD3-syn CPH (FRa_CPH), FRa-CD3-syn CH (FRa_CH), FRa-CD3-syn CP (FRa_CP), FRa_CD3_syn CNR2 (FRa_C), FRa-CD3-syn HCAR2 (FRa_H) and FRa-CD3-syn P2Y14 (FRa_P) exhibited a cytotoxic effect (over 80% killing activity) on Ovcar4 cancer cells.

### Example 2 In Vivo Activity

In this example, the anti-tumor effect of the present engineered T cells was evaluated by an animal model. As described in “Materials and Methods” of the present disclosure, the OVCAR-3 cells were inoculated subcutaneously into the flank of mice. When the average size of tumor reached about 100 mm³, the FRa_CD3_syn CPH-T or FRa-TAC-T cells (0.3×10⁶ or 1.5×10⁶) were intravenously (i.v.) administered to the tumor-bearing mice.

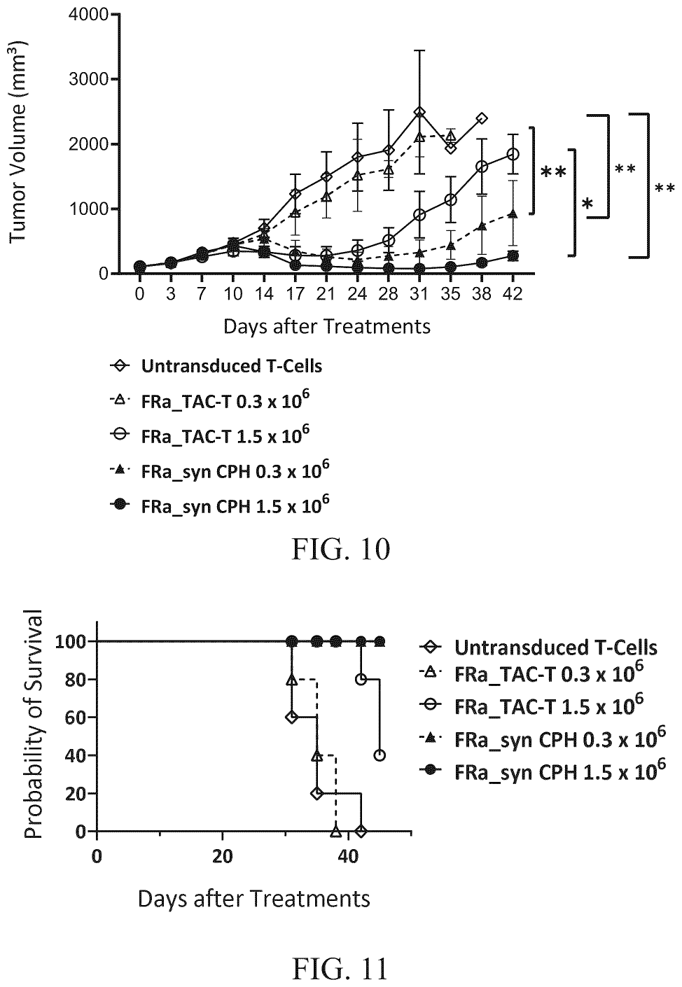

The data of FIGS. 10 and 11 demonstrated that compared to the positive control FRa-TAC-T cells, the administration of the present FRa_CD3_syn CPH-T cells greatly inhibited tumor growth (FIG. 10) and prolonged the survival rate of tumor-bearing mice (FIG. 11).

In conclusion, the present disclosure provides several TM/IC domains, each of which is useful in conjugating to one or more scFvs/peptides, and expressing the scFv(s)/peptide(s) on cell membrane of cells (e.g., T cells). According to the examples of the present disclosure, the engineered T cells expressing the TM/IC-based polypeptides exhibited targeting and cytotoxic effects on cancer cells, and thus provide a potential means to treat cancers.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant polypeptide comprising a first bi-functional domain, and a first single-chain fragment variable (scFv) or a peptide linked to the N-terminus of the first bi-functional domain, wherein
the first bi-functional domain comprises, in sequence, an intracellular loop 1 (ICL1), an ICL2, an ICL3 and a C-terminal region of a first G protein-coupled receptor (GPCR), and is characterized in not having an extracellular domain nor a transmembrane domain of the first GPCR.

2. The recombinant polypeptide of claim 1, wherein the first bi-functional domain consists of the ICL1, ICL2, ICL3 and C-terminal region of the first GPCR.

3. The recombinant polypeptide of claim 1, wherein the first GPCR is a class A GPCR.

4. The recombinant polypeptide of claim 3, wherein the first GPCR is cannabinoid receptor 2 (CNR2), hydroxycarboxylic acid receptor 2 (HCAR2), G-protein coupled receptor 84 (GPR84), or P2Y purinoceptor 14 (P2Y14).

5. The recombinant polypeptide of claim 4, wherein the first GPCR is the CNR2, and the first bi-functional domain comprises the amino acid sequence of SEQ ID NO: 2.

6. The recombinant polypeptide of claim 4, wherein the first GPCR is the HCAR2, and the first bi-functional domain comprises the amino acid sequence of SEQ ID NO: 4.

7. The recombinant polypeptide of claim 4, wherein the first GPCR is the GPR84, and the first bi-functional domain comprises the amino acid sequence of SEQ ID NO: 6.

8. The recombinant polypeptide of claim 4, wherein the first GPCR is the P2Y14, and the first bi-functional domain comprises the amino acid sequence of SEQ ID NO: 8.

9. The recombinant polypeptide of claim 1, wherein the first scFv is specific to CD3 or NKp46.

10. The recombinant polypeptide of claim 1, further comprising a second scFv that is linked to the N-terminus of the first scFv or peptide, and is specific to a tumor-associated antigen (TAA).

11. The recombinant polypeptide of claim 1, further comprising a second bi-functional domain disposed at and connected to the C-terminus of the first bi-functional domain, wherein
the second bi-functional domain comprises in sequence, an ICL1, an ICL2, an ICL3 and a C-terminal region of a second GPCR;
the second bi-functional domain is characterized in not having an extracellular domain nor a transmembrane domain of the second GPCR; and
the second GPCR is different from the first GPCR.

12. The recombinant polypeptide of claim 11, wherein the second bi-functional domain consists of the ICL1, ICL2, ICL3 and C-terminal region of the second GPCR.

13. The recombinant polypeptide of claim 11, wherein each of the first and second GPCRs is a class A GPCR.

14. The recombinant polypeptide of claim 13, wherein the first and second GPCRs are independently selected from the group consisting of CNR2, HCAR2, GPR84 and P2Y14.

15. The recombinant polypeptide of claim 14, wherein
the first and second GPCRs are respectively the CNR2 and P2Y14; and
the first and second bi-functional domains respectively comprise the amino acid sequences of SEQ ID NOs: 2 and 8.

16. The recombinant polypeptide of claim 14, wherein
the first and second GPCRs are respectively the CNR2 and HCAR2; and
the first and second bi-functional domains respectively comprise the amino acid sequences of SEQ ID NOs: 2 and 4.

17. The recombinant polypeptide of claim 11, further comprising a third bi-functional domain disposed at and connected to the C-terminus of the second bi-functional domain, wherein
the third bi-functional domain comprises, in sequence, an ICL1, an ICL2, an ICL3 and a C-terminal region of a third GPCR;
the third bi-functional domain is characterized in not having an extracellular domain nor a transmembrane domain of the third GPCR; and
the first, second and third GPCRs are different from one another.

18. The recombinant polypeptide of claim 17, wherein the third bi-functional domain consists of the ICL1, ICL2, ICL3 and C-terminal region of the third GPCR.

19. The recombinant polypeptide of claim 17, wherein each of the first, second and third GPCRs is a class A GPCR.

20. The recombinant polypeptide of claim 17, wherein the first, second and third GPCRs are independently selected from the group consisting of CNR2, HCAR2, GPR84 and P2Y14.

21. The recombinant polypeptide of claim 20, wherein
the first, second and third GPCRs are respectively the CNR2, P2Y14 and HCAR2; and
the first, second and third bi-functional domains respectively comprise the amino acid sequences of SEQ ID NOs: 2, 8 and 4.

22. A recombinant nucleic acid encoding the recombinant polypeptide of claim 1, comprising a promoter, and a first and a second coding sequences operably linked to the promoter, wherein the first coding sequence encodes the first single-chain fragment variable (scFv) or the peptide, and the second coding sequence is disposed downstream to the first coding sequence and encodes the first bi-functional domain.

23. The recombinant nucleic acid of claim 22, further comprising a third coding sequence operably linked to the promoter and is disposed upstream to the first coding sequence, wherein the third coding sequence encodes a second scFv specific to a tumor-associated antigen (TAA).

24. The recombinant nucleic acid of claim 22, wherein the second coding sequence further encodes a second bi-functional domain disposed at and connected to the C-terminus of the first bi-functional domain, wherein
the second bi-functional domain comprises in sequence, an ICL1, an ICL2, an ICL3 and a C-terminal region of a second GPCR;
the second bi-functional domain is characterized in not having an extracellular domain nor a transmembrane domain of the second GPCR; and
the second GPCR is different from the first GPCR.

25. The recombinant nucleic acid of claim 24, wherein the second coding sequence further encodes a third bi-functional domain disposed at and connected to the C-terminus of the second bi-functional domain, wherein
the third bi-functional domain comprises, in sequence, an ICL1, an ICL2, an ICL3 and a C-terminal region of a third GPCR;
the third bi-functional domain is characterized in not having an extracellular domain nor a transmembrane domain of the third GPCR; and
the first, second and third GPCRs are different from one another.

26. A method of treating a cancer in a subject, comprising administering to the subject an effective amount of an immune cell expressing the recombinant polypeptide of claim 1.
